(19) United States
(12) Patent Application Publication
(10) Pub. No.: US 2025/0099407 A1
(43) Pub. Date: Mar. 27, 2025

(54) 2-AMINO BENZOPHENONE DERIVATIVES FOR TARGETED FERROPTOSIS INDUCTION IN CANCER THERAPY

(71) Applicant: INDIAN INSTITUTE OF TECHNOLOGY KANPUR, KANPUR (IN)

(72) Inventors: DHARMARAJA ALLIMUTHU, Kanpur (IN); DHARMENDRA K. YADAV, Kanpur (IN); SONA TIWARI, Kanpur (IN); SHWETA KHANNA, Kanpur (IN); SAI KUMARI VECHALAPU, Kanpur (IN)

(21) Appl. No.: 18/890,836

(22) Filed: Sep. 20, 2024

(30) Foreign Application Priority Data
Sep. 22, 2023 (IN) 202311063839

(51) Int. Cl. A61K 31/18; A61K 31/167; A61K 31/381; C07C 235/78; C07C 311/13; C07D 333/36

(52) U.S. Cl. CPC A61K 31/18; A61K 31/167; A61K 31/381; C07C 235/78; C07C 311/13; C07D 333/36

(57) ABSTRACT

The present invention discloses a novel class of covalent small molecules comprising 2-amino benzophenone derivatives for targeted ferroptosis induction in cancer cells. The present invention also includes strategies for target identification, hit-to-lead optimization, and combination therapies to enhance therapeutic efficacy. A synthesis and characterization of a library of unique organic structural scaffolds encompassing natural/unnatural amino acids, aromatic and heterocyclic linkers coupled with electrophilic units possessing diverse nucleophile reactivity profiles is provided. The molecules exhibit sub-micromolar to nanomolar inhibitory potency (IC₅₀) when screened against a panel of cancer cell lines including MCF7 (breast cancer), MDA-MB-231 (triple negative breast cancer), 22Rv1 (Prostate), PC3, (prostate cancer), Jurkat J6 (acute T-cell Leukaemia), RPMI 8226 (B lymphocytes), U87MG (glioblastoma), HCT-116 (colon cancer) and A375 (melanoma)) and kidney cells (HEK293). The molecules exhibit potent antiproliferative effects across various cancer cell lines, offering a promising alternative to conventional chemotherapies with reduced side effects.

### EARLIEST PRIORITY DATE

This Application claims priority from a Complete patent application filed in IN having Patent Application No. 202311063839, filed on Sep. 22, 2023, and titled “2-AMINO BENZOPHENONE DERIVATIVES FOR TARGETED FERROPTOSIS INDUCTION IN CANCER THERAPY”.

### FIELD OF INVENTION

Embodiment of the present invention relates to 2-aminobenzophenone derivatives for a targeted ferroptosis induction in cancer therapy. More particularly, the present invention relates to identification and optimization of covalent small molecules comprising 2-aminobenzophenone derivatives designed for selectively triggering ferroptosis in cancer cells for treatment of cancer.

### BACKGROUND

Covalent small molecules emerged as a promising tool for deciphering protein functions and reliable therapeutics. Owing to their incredible efficacy, covalent modifiers are emerging as a powerful chemical modality for the treatment of cancers as advocated by the FDA approval of several covalent small molecule inhibitors including the recently approved (2022) KRAS oncogene inhibitor, Sotorasib as disclosed in Dagogo et. al., 2017. In hindsight, the concern of covalent small molecules engaging idiosyncratic off-target proteins rise the dilemma of their safety profiles as therapeutic candidates and in vivo probes. The quality of covalent chemical probes or therapeutics is dictated majorly by the electrophiles' selective reactivity to a specific amino acid residue within the target protein as disclosed in Koren et. al., 2021. Completely silencing the drug target is a powerful quality of covalent modifications, essentially permanent and durable target occupancy is a key contributor to the molecules' potency. On the back of the success of several FDA-approved covalent therapeutics, rationally designed chemical scaffolds assisted by advanced technological tools enable a precise design of target-based small molecule therapeutics development. Target-based therapeutics are a big success in the recent past, particularly, targeted therapeutics market size is expected to reach USD $162.89 billion in 2028 ($93 billion USD in 2021). For example, ibrutinib, a Bruton tyrosine kinase inhibitor used for the treatment of blood cancer and immune cell cancer is sold for $21 billion USD between 2018 to 2021 and the projected sale is $60B USD by 2030. Together, Covalent therapeutics development is in vogue owing to the rapid identification of drug/off-targets and heightened potency over the non-covalent counterparts. A large majority of anticancer therapeutics that function by covalent protein modification are known modifiers of cysteine active sites in protein kinases. In this invention disclosure, we report a library of cysteine-reactive covalent therapeutics that operate by ferroptosis to induce cell death in cancer cells. Ferroptosis is a programmed cell death process activated under pathological conditions and not found in healthy cells.

Identification of unique druggable targets in cancer cells is strongly encouraged considering the high rate of therapeutic tolerance in cancer cells to the existing cluster of FDA-approved drugs. Particularly small molecules operating through a well-defined mechanism of action are advantageous due to their tractable idiosyncratic actions.

Global cancer incidences continue to rise with 20 million new cases and 10 million deaths in 2020 alone. Importantly, the emergence of drug resistance in cancer patients to the majority of the existing chemotherapeutics warrants continued efforts for the identification of new therapeutic targets and drugs. While several FDA-approved anticancer therapeutics target apoptosis, a programmed cell death pathway, it is also a natural cell death process in healthy cells that results in undesired toxicological effects. Ferroptosis is a mode of programmed cell death found to be activated under pathological conditions and not in healthy cells as disclosed in Yan et. al., 2021, Li et. al., 2020, and Dixon et. al., 2012. Therefore, it is safe to assume investing our efforts in developing structurally unique small molecule-based ferroptosis inducers and identification of new protein targets linked to ferroptosis in cancer cells would be highly beneficial. Ferroptosis is induced by multiple pathways, majorly by lipid peroxidation leading to the accumulation of reactive oxygen species (ROS) as disclosed in Chen et. al., 2021, Chen et. al., 2023, Du et. al., 2022, Naveen Kumar et. al., 2018, and Shimada et. al., 2016. This ROS buildup can damage various biomacromolecules including DNA, lipids, cell membranes, and proteins leading to oxidative stress and cell death as disclosed in Naveen Kumar et. al., 2019. Iron buildup in the cells is another mode of ferroptosis induction where the free iron pool can do the Fenton reaction with peroxides to promote radical-ROS and oxidative stress. Further, glutathione is a thiol-containing tripeptide found in millimolar concentrations in cells that act as an antioxidant to prevent cells from oxidative cell death. Depletion of glutathione levels is also well known to promote ferroptosis. Therefore, targeting the mechanisms that promote the accumulation of lipid peroxides could lead to the induction of ferroptosis.⁴ Glutathione peroxidase 4 (GPX4) is an antioxidant selenocysteine-protein, critical in regulating lipid peroxides in cells to control oxidative stress. Inhibition of GPX4 by a small molecule, RSL3 triggers ferroptosis in cancer cells. Ferroptosis suppressor protein 1 (FSP1) is another target found to deplete lipid peroxides and suppress ferroptosis independent of GPX4. Further, fatty acids metabolism regulator, ACSL4 has been shown to modulate lipid peroxides accumulation and promote oxidative stress in cells to regulate ferroptosis. Inhibition of transmembrane cysteine-glutamate transporter, SLC7A11 is another mechanism of depleting glutathione biosynthesis to trigger ferroptosis as disclosed in Kagan et. al., 2017, Eaton et. al., 2020, and Bersuker et. al., 2019.

The prior arts reported heretofore are having several disadvantages that only a handful of protein targets and fewer mechanism-based ferroptosis inducers are available. Developing any structurally unique class of ferroptosis inducers and/or identification of new therapeutic targets that modulate ferroptosis is strongly encouraged.

Therefore, there is a need for a screening of covalent small molecules library for the identification of a unique class of 2-aminobenzophenone capable of alkylating thiols of cysteines indicating modification of catalytic cysteine residues in target protein to inhibit function of the target protein and covalent modifiers of cysteine-containing proteins linked to ferroptosis in cancer cells. There is need for thiol-reactivity-based covalent small molecules capable of inducing ferroptosis as a design strategy for identification of potential chemical scaffolds for anticancer drug development.

### SUMMARY

Embodiments of the present invention relate to development of covalent small molecules comprising 2-aminobenzophenone derivatives designed for a targeted induction of ferroptosis in cancer cells as a novel approach for cancer therapy. The compounds exhibit potent antiproliferative effects on various cancer cell lines, making them promising candidates for cancer therapy.

In an embodiment of the present invention, 2-aminobenzophenone derivatives of Formula I are provided. The 2-aminobenzophenone derivatives of Formula I,

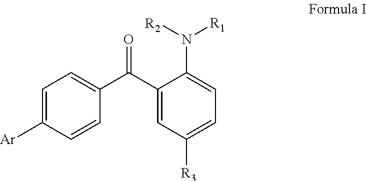

wherein,
R₁ is selected from a group consisting one of —CH₃,

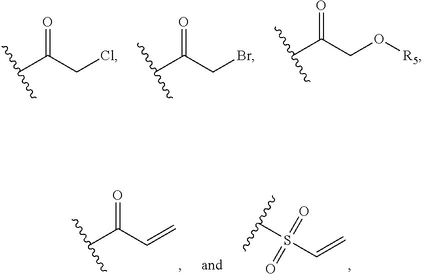

wherein R₅ is selected from a group consisting one of phenyl, 4-fluorophenyl, 4-bromophenyl, 4-tert-butylphenyl, and pentaflurophenyl;
R₂ is selected from a group consisting one of —H, methyl, allyl, benzyl, isopropyl, and propargyl;
R₃ is selected from a group consisting one of —H, —NO₂,

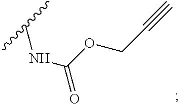 [SMILES: *NC(=O)OCC#C]

and
Ar is selected from a group consisting one of

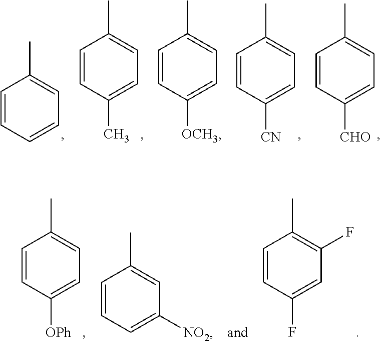

In another embodiment of the present invention, a pharmaceutical composition for treatment of cancer is provided. The pharmaceutical composition comprising one or more of the 2-aminobenzophenone derivatives of Formula I, pharmaceutically acceptable salts thereof, and one of a pharmaceutically acceptable carrier, and an excipient. The pharmaceutical composition is configured for inducing ferroptosis in cancer cells.

In another embodiment of the present invention, a process for the preparation of the 2-aminobenzophenone derivatives of the Formula I is provided. The process begins with reacting 2-amino-benzophenone 1 with alkyl halide (R₆—X) in presence of 3 equivalent (equiv.) potassium carbonate (K₂CO₃) in dimethyl formamide at 90° C. for 12 hours to obtain an intermediate 2-5. R₆ is selected from a group consisting of methyl 2, allyl 3, benzyl 4, and propargyl 5. The intermediate 2-5 is reacted with 1.5 equiv. of chloroacetyl chloride in presence of 3 equiv. sodium bicarbonate (NaHCO₃) carbonate in dichloromethane at room temperature for 6 hours to obtain one of the 2-aminobenzophenone derivatives of Formula I consisting one of N-(2-benzoylphenyl)-2-chloro-N-methylacetamide (IITK3001), N-allyl-N-(2-benzoylphenyl)-2-chloroacetamide (IITK3018), N-(2-benzoylphenyl)-N-benzyl-2-chloroacetamide (IITK3034), and N-(2-benzoylphenyl)-2-chloro-N-(prop-2-yn-1-yl)acetamide (IITK3003)

To further clarify the advantages and features of the present invention, a more particular description of the invention will follow by reference to specific embodiments thereof, which are illustrated in the appended figures. It is to be appreciated that these figures depict only typical embodiments of the invention and are therefore not to be considered limiting in scope. The invention will be described and explained with additional specificity and detail with the appended figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The disclosure will be described and explained with additional specificity and detail with the accompanying figures in which:

FIG. 1 depicts a schematic representation of synthesis of N-(2-benzoylphenyl)-2-Chloro-N-alkylacetamide (IITK3001 and derivatives), in accordance with an embodiment of the present invention;

FIG. 2 depicts a schematic representation of synthesis of N-(2-benzoylphenyl)-2-Bromo-N-alkylacetamide (IITK3002 and derivatives), in accordance with an embodiment of the present invention;

FIG. 3 depicts a schematic representation of synthesis of N-(2-benzoylphenyl)-2-Chloro-N-Isopropylacetamide (IITK3028), in accordance with an embodiment of the present invention;

FIG. 4 depicts a schematic representation of synthesis of N-(2-benzoylphenyl)-N-alkyl-2-phenoxyacetamide derivatives, in accordance with an embodiment of the present invention;

FIG. 5 depicts a schematic representation of synthesis of N-(2-benzoyl-4-nitrophenyl)-2-chloro-N-methylacetamide, in accordance with an embodiment of the present invention;

FIG. 6 depicts a schematic representation of synthesis of N-methyl 2-aminobenzophenone with different electrophile, in accordance with an embodiment of the present invention;

FIG. 7 depicts a schematic representation of synthesis of 2-halo-N-(4-methoxybenzyl) acetamide, in accordance with an embodiment of the present invention;

FIG. 8 depicts a schematic representation of synthesis of [1,1′-biphenyl]-4-yl(2-(methylamino)phenyl)methanone, in accordance with an embodiment of the present invention;

FIG. 9 depicts a schematic representation of synthesis of N-(2-([1,1′-biphenyl]-4-carbonyl)phenyl)-N-methylacetamide derivatives, in accordance with an embodiment of the present invention;

FIG. 10 depicts major covalent drug development timeline as disclosed in a prior art Boike et. al., 2022;

FIG. 11 depicts screening of electrophilic small molecules has identified IITK3001 and IITK1037 as potent inhibitors of Huh-7 cells, in accordance with an embodiment of the present invention;

FIG. 12 depicts IITK3001 dose-dependently inhibited the proliferation of twelve-different cancer cell lines, in accordance with an embodiment of the present invention;

FIG. 13 depicts IITK3002 dose-dependently inhibited the proliferation of twelve-different cancer cell lines, in accordance with an embodiment of the present invention;

FIG. 14 depicts a combination experiment indicates the involvement of thiol modulation by IITK3001/IITK3002 on the viability of Huh-7 and 4T1 cells, in accordance with an embodiment of the present invention;

FIG. 15 depicts an intracellular ROS generation by IITK3001/IITK3002 and its quenching by the combination of NAC captured using H2-DCFDA oxidation assay in Huh-7 cells, in accordance with an embodiment of the present invention;

FIG. 16 depicts ferroptosis induction as a cell death mechanism is established for IITK3001 using combination experiments with ferroptosis inducers and inhibitors, in accordance with an embodiment of the present invention;

FIG. 17 depicts IITK3001-mediated ferroptosis induction as a cell death mechanism in 4T1 cells is recorded, in accordance with an embodiment of the present invention;

FIG. 18 depicts ferroptosis induction as a cell death mechanism is established for IITK3002 using combination experiments with ferroptosis inducers and inhibitors, in accordance with an embodiment of the present invention;

FIG. 19 depicts IITK3001-mediated ferroptosis induction as a cell death mechanism in 4T1 cells is recorded, in accordance with an embodiment of the present invention;

FIG. 20 depicts combining apoptosis and necroptosis inhibitors ruled out the possibility of respective cell death mechanisms upon treatment with IITK3001 or IITK3002 in Huh-7 and 4T1 cells, in accordance with an embodiment of the present invention;

FIG. 21 depicts cell viability assessment of alkyne-versions (IITK3003 and IITK3004) confirmed retaining the potency in Huh-7 cells and in-gel fluorescence analysis revealed a single major band indicating a selective protein modification, in accordance with an embodiment of the present invention;

FIG. 22 depicts ferroptosis induction was established for IITK3003 using combination experiments in Huh-7 cells, in accordance with an embodiment of the present invention;

FIG. 23 depicts ferroptosis induction is established for IITK3003 using combination experiments in 4T1 cells, in accordance with an embodiment of the present invention;

FIG. 24 depicts ferroptosis induction was established for IITK3004 using combination experiments in Huh-7 cells, in accordance with an embodiment of the present invention; and

FIG. 25 depicts ferroptosis induction is established for IITK3004 using combination experiments in 4T1 cells, in accordance with an embodiment of the present invention.

Further, those skilled in the art will appreciate that elements in the figures are illustrated for simplicity and may not have necessarily been drawn to scale. Furthermore, in terms of the method steps, chemical compounds, and parameters used herein may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more components, compounds, and ingredients preceded by “comprises . . . a” does not, without more constraints, preclude the existence of other components or compounds or ingredients or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present invention relates to development of covalent small molecules comprising 2-aminobenzophenone derivatives designed for a targeted induction of ferroptosis in cancer cells as a novel approach for cancer therapy. The compounds exhibit potent antiproliferative effects on various cancer cell lines, making them promising candidates for cancer therapy.

In an embodiment of the present invention, 2-aminobenzophenone derivatives of Formula I is provided. The 2-aminobenzophenone derivatives of Formula I,

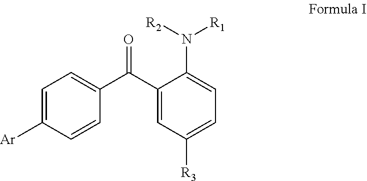

wherein,
R₁ is selected from a group consisting one of —CH₃,

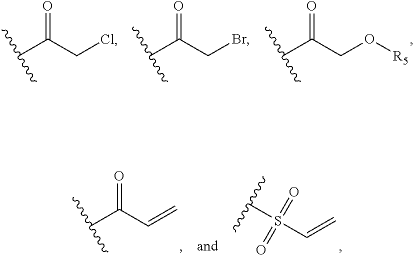

wherein R₅ is selected from a group consisting one of phenyl, 4-fluorophenyl, 4-bromophenyl, 4-tert-butylphenyl, and pentaflurophenyl;
R₂ is selected from a group consisting one of —H, methyl, allyl, benzyl, isopropyl, and propargyl;

R₃ is selected from a group consisting one of —H, —NO₂,

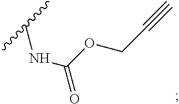 [SMILES: *NC(=O)OCC#C]

and
Ar is selected from a group consisting one of

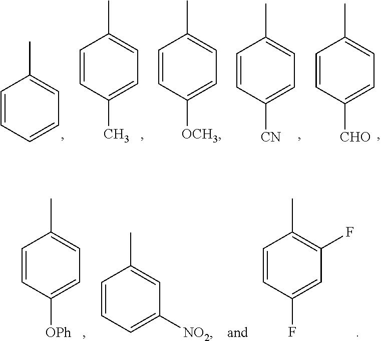

In another embodiment of the present invention, the 2-aminobenzophenone derivatives of Formula I are selected from a group consisting of N-(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), N-(2-benzoylphenyl)-2-bromo-N-(prop-2-yn-1-yl)acetamide (IITK3004), N-(2-benzoylphenyl)-N-methyl-2-phenoxyacetamide (IITK3005), N-(2-benzoylphenyl)-2-(4-fluorophenoxy)-N-methylacetamide (IITK3006), N-(2-benzoylphenyl)-N-methyl-2-(perfluorophenoxy)acetamide (IITK3007), N-(2-benzoylphenyl)-2-(4-(tert-butyl)phenoxy)-N-methylacetamide (IITK3008), N-(2-benzoylphenyl)-2-(4-bromophenoxy)-N-methylacetamide (IITK3009), N-(2-benzoylphenyl)-N-methylacrylamide (IITK3010), N-(2-benzoylphenyl)-N-methylethenesulfonamide (IITK3011), N-(2-benzoylphenyl)-2-chloroacetamide (IITK3012), N-(2-benzoylphenyl)-2-bromoacetamide (IITK3013), N-(2-benzoylphenyl)-2-phenoxyacetamide (IITK3014), N-allyl-N-(2-benzoylphenyl)-2-chloroacetamide (IITK3018), N-allyl-N-(2-benzoylphenyl)-2-bromoacetamide (IITK3019), N-allyl-N-(2-benzoylphenyl)-2-phenoxyacetamide (IITK3020), N-allyl-N-(2-benzoylphenyl)-2-(4-fluorophenoxy)acetamide (IITK3021), N-allyl-N-(2-benzoylphenyl)-2-(perfluorophenoxy)acetamide (IITK3022), N-allyl-N-(2-benzoylphenyl)-2-(4-(tert-butyl)phenoxy)acetamide (IITK3023), N-allyl-N-(2-benzoylphenyl)-2-(4-bromophenoxy)acetamide (IITK3024), N-allyl-N-(2-benzoylphenyl)-2-(4-nitrophenoxy)acetamide (IITK3025), N-allyl-N-(2-benzoylphenyl)acrylamide (IITK3026), N-allyl-N-(2-benzoylphenyl)ethenesulfonamide (IITK3027), N-(2-benzoylphenyl)-2-chloro-N-isopropylacetamide (IITK3028), N-(2-benzoylphenyl)-2-phenoxy-N-(prop-2-yn-1-yl)acetamide (IITK3029), N-(2-benzoylphenyl)-2-(4-fluorophenoxy)-N-(prop-2-yn-1-yl)acetamide (IITK3030), N-(2-benzoylphenyl)-2-(perfluorophenoxy)-N-(prop-2-yn-1-yl)acetamide (IITK3031), N-(2-benzoylphenyl)-2-(4-(tert-butyl)phenoxy)-N-(prop-2-yn-1-yl)acetamide (IITK3032), N-(2-benzoylphenyl)-2-(4-bromophenoxy)-N-(prop-2-yn-1-yl)acetamide (IITK3033), N-(2-benzoylphenyl)-N-benzyl-2-chloroacetamide (IITK3034), N-(2-benzoylphenyl)-N-benzyl-2-bromoacetamide (IITK3035), N-(2-benzoylphenyl)-N-benzyl-2-phenoxyacetamide (IITK3036), N-(2-benzoylphenyl)-N-benzyl-2-(4-fluorophenoxy)acetamide (IITK3037), N-(2-benzoylphenyl)-N-benzyl-2-(4-(tert-butyl)phenoxy)acetamide (IITK3038), N-(2-benzoyl-4-nitrophenyl)-2-bromoacetamide (IITK3051), prop-2-yn-1-yl (3-benzoyl-4-(2-chloro-N-methylacetamido)phenyl)carbamate (IITK3052), prop-2-yn-1-yl (3-benzoyl-4-(2-bromo-N-methylacetamido)phenyl)carbamate (IITK3053), N-(3-benzoyl-4,5-dimethylthiophen-2-yl)-2-chloroacetamide (IITK3054), N-(2-([1,1′-biphenyl]-4-carbonyl)phenyl)-2-chloro-N-methylacetamide (IITK3055), N-(2-([1,1′-biphenyl]-4-carbonyl)phenyl)-2-bromo-N-methylacetamide (IITK3056), 2-bromo-N-methyl-N-(2-(4′-methyl-[1,1′-biphenyl]-4-carbonyl)phenyl)acetamide (IITK3057), 2-chloro-N-(2-(4′-formyl-[1,1′-biphenyl]-4-carbonyl)phenyl)-N-methylacetamide (IITK3058), 2-bromo-N-(2-(4′-formyl-[1,1′-biphenyl]-4-carbonyl)phenyl)-N-methylacetamide (IITK3059), 2-chloro-N-(2-(4′-cyano-[1,1′-biphenyl]-4-carbonyl)phenyl)-N-methylacetamide (IITK3060), 2-bromo-N-(2-(4′-cyano-[1,1′-biphenyl]-4-carbonyl)phenyl)-N-methylacetamide (IITK3061), 2-chloro-N-methyl-N-(2-(3′-phenoxy-1[1,1′-biphenyl]-4-carbonyl)phenyl)acetamide (IITK3062), 2-bromo-N-methyl-N-(2-(3′-phenoxy-[1,1′-biphenyl]-4-carbonyl)phenyl)acetamide (IITK3063), 2-chloro-N-(2-(4′-methoxy-[1,1′-biphenyl]-4-carbonyl)phenyl)-N-methylacetamide (IITK3064), 2-bromo-N-(2-(4′-methoxy-[1,1′-biphenyl]-4-carbonyl)phenyl)-N-methylacetamide (IITK3065), 2-chloro-N-(2-(2′,4′-difluoro-[1,1′-biphenyl]-4-carbonyl)phenyl)-N-methylacetamide (IITK3066), 2-bromo-N-(2-(2′,4′-difluoro-[1,1′-biphenyl]-4-carbonyl)phenyl)-N-methylacetamide (IITK3067), 2-chloro-N-methyl-N-(2-(3′-nitro-[1,1′-biphenyl]-4-carbonyl)phenyl)acetamide (IITK3068), 2-bromo-N-methyl-N-(2-(3′-nitro-[1,1′-biphenyl]-4-carbonyl)phenyl)acetamide (IITK3069), N-(2-([1,1′-biphenyl]-4-carbonyl)phenyl)-N-methylethenesulfonamide (IITK3070), N-(2-([1,1′-biphenyl]-4-carbonyl)phenyl)-2-bromo-N-(prop-2-yn-1-yl)acetamide (IITK3071), and N-(2-([1,1′-biphenyl]-4-carbonyl)phenyl)-2-chloro-N-(prop-2-yn-1-yl)acetamide (IITK3072), and N-(2-benzoyl-4-nitrophenyl)-2-chloro-N-methylacetamide (IITK3073).

In another embodiment of the present invention, the 2-aminobenzophenone derivatives of Formula I is selected from a group consisting one of N-(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), and N-(2-benzoylphenyl)-2-bromo-N-(prop-2-yn-1-yl)acetamide (IITK3004).

In another embodiment of the present invention, a pharmaceutical composition for treatment of cancer is provided. The pharmaceutical composition comprising one or more of the 2-aminobenzophenone derivatives of Formula I, pharmaceutically acceptable salts thereof, and one of a pharmaceutically acceptable carrier, and an excipient. The pharmaceutical composition is configured for inducing ferroptosis in cancer cells. The 2-aminobenzophenone derivatives of Formula I is selected from a group consisting one of -(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), and N-(2-benzoylphenyl)-2-bromo-N-(prop-2-yn-1-yl)acetamide (IITK3004).

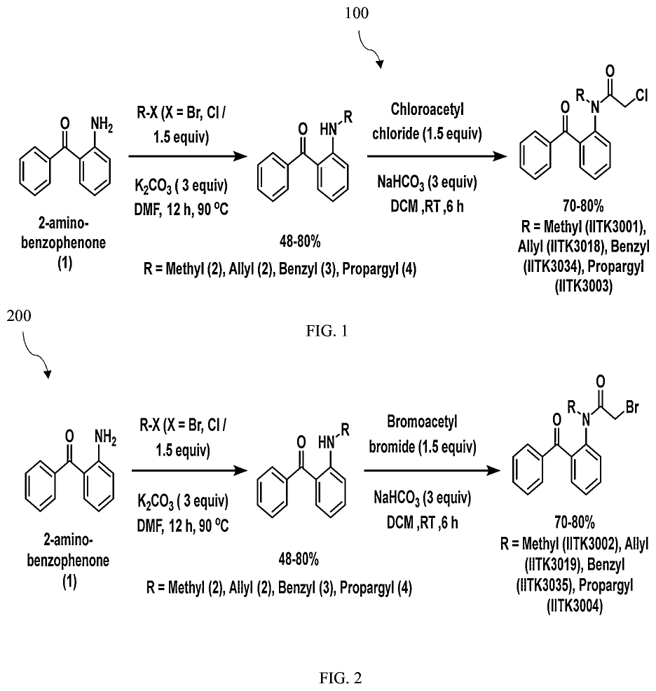

FIG. 1 depicts a schematic representation of synthesis of N-(2-benzoylphenyl)-2-Chloro-N-alkylacetamide (IITK3001 and derivatives), in accordance with an embodiment of the present invention.

FIG. 2 depicts a schematic representation of synthesis of N-(2-benzoylphenyl)-2-Bromo-N-alkylacetamide (IITK3002 and derivatives), in accordance with an embodiment of the present invention.

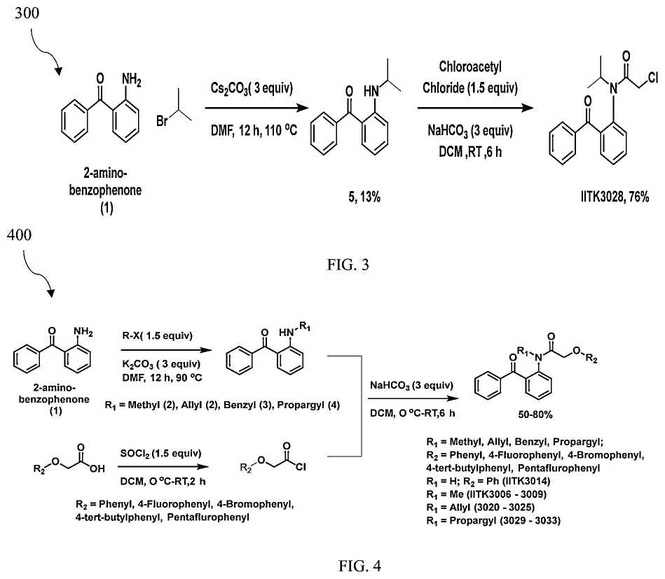

FIG. 3 depicts a schematic representation of synthesis of N-(2-benzoylphenyl)-2-Chloro-N-Isopropylacetamide (IITK3028), in accordance with an embodiment of the present invention.

FIG. 4 depicts a schematic representation of synthesis of N-(2-benzoylphenyl)-N-alkyl-2-phenoxyacetamide derivatives, in accordance with an embodiment of the present invention.

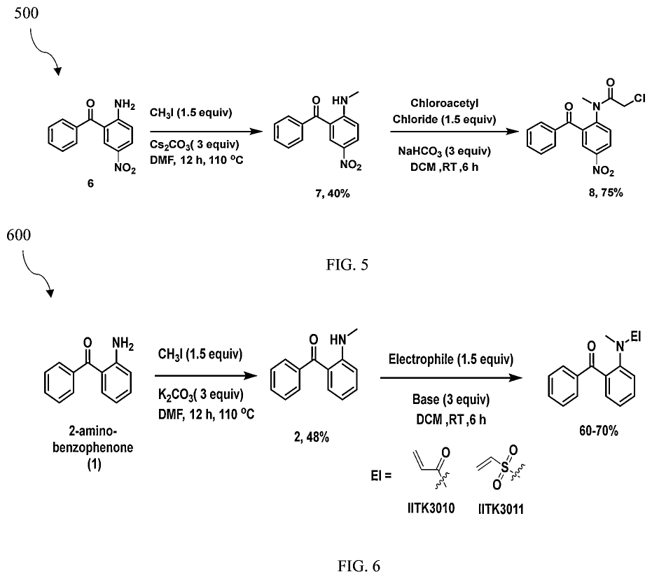

FIG. 5 depicts a schematic representation of synthesis of N-(2-benzoyl-4-nitrophenyl)-2-chloro-N-methylacetamide, in accordance with an embodiment of the present invention.

FIG. 6 depicts a schematic representation of synthesis of N-methyl 2-aminobenzophenone with different electrophile, in accordance with an embodiment of the present invention.

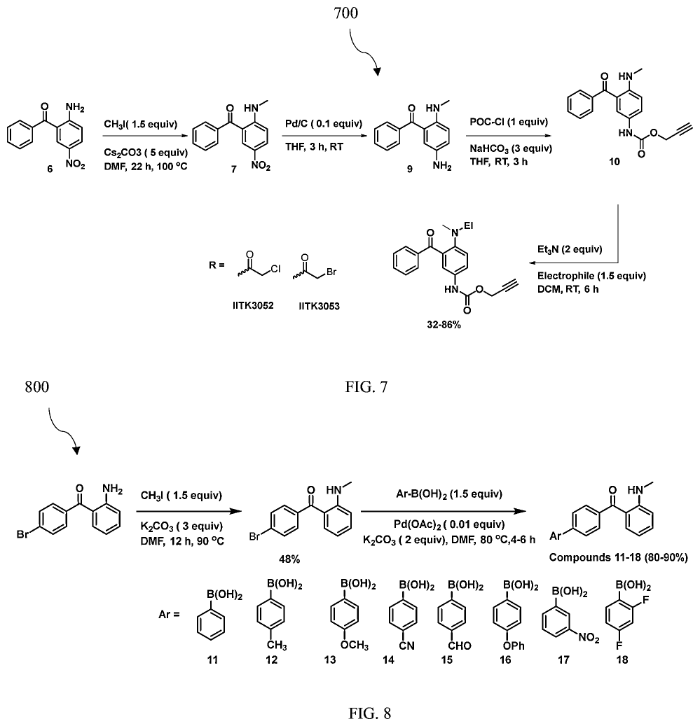

FIG. 7 depicts a schematic representation of synthesis of 2-halo-N-(4-methoxybenzyl) acetamide, in accordance with an embodiment of the present invention.

FIG. 8 depicts a schematic representation of synthesis of [1,1′-biphenyl]-4-yl(2-(methylamino)phenyl)methanone, in accordance with an embodiment of the present invention.

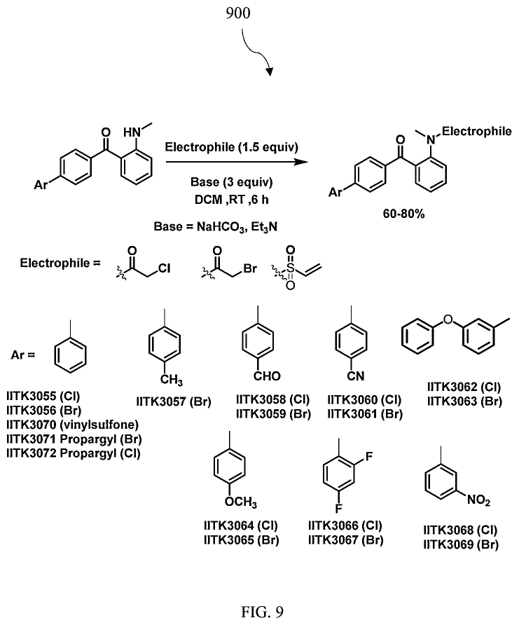

FIG. 9 depicts a schematic representation of synthesis of N-(2-([1,1′-biphenyl]-4-carbonyl)phenyl)-N-methylacetamide derivatives, in accordance with an embodiment of the present invention.

In another embodiment of the present invention, a process for the preparation of the 2-aminobenzophenone derivatives of the Formula I is provided. The process begins with reacting 2-amino-benzophenone 1 with alkyl halide (R₆—X) in presence of 3 equivalent (equiv.) potassium carbonate (K₂CO₃) in dimethyl formamide at 90° C. for 12 hours to obtain an intermediate 2-5. R₆ is selected from a group consisting of methyl 2, allyl 3, benzyl 4, and propargyl 5. The intermediate 2-5 is reacted with 1.5 equiv. of chloroacetyl chloride in presence of 3 equiv. sodium bicarbonate (NaHCO₃) carbonate in dichloromethane at room temperature for 6 hours to obtain one of the 2-aminobenzophenone derivatives of Formula I consisting one of N-(2-benzoylphenyl)-2-chloro-N-methylacetamide (IITK3001), N-allyl-N-(2-benzoylphenyl)-2-chloroacetamide (IITK3018), N-(2-benzoylphenyl)-N-benzyl-2-chloroacetamide (IITK3034), and N-(2-benzoylphenyl)-2-chloro-N-(prop-2-yn-1-yl)acetamide (IITK3003).

In another embodiment of the present invention, the process comprises reacting 2-amino-benzophenone 1 with alkyl halide (R₆—X) in presence of 3 equivalent (equiv.) potassium carbonate (K₂CO₃) in dimethyl formamide at 90° C. for 12 hours to obtain the intermediate 2-5. R₆ is selected from a group consisting of methyl 2, allyl 3, benzyl 4, and propargyl 5. The intermediate 2-5 is reacted with 1.5 equiv. of bromoacetyl chloride in presence of 3 equiv. sodium bicarbonate (NaHCO₃) in dichloromethane at room temperature for 6 hours to obtain one of the 2-aminobenzophenone derivatives of Formula I consisting one of N-(2-benzoylphenyl)-2-bromo-N-methylacetamide (IITK3002), N-allyl-N-(2-benzoylphenyl)-2-bromoacetamide (IITK3019), N-(2-benzoylphenyl)-N-benzyl-2-bromoacetamide (IITK3035), and N-(2-benzoylphenyl)-2-bromo-N-(prop-2-yn-1-yl)acetamide (IITK3004).

In another embodiment of the present invention, the process comprises reacting 2-amino-benzophenone 1 with isopropyl bromide in presence of 3 equiv. cesium carbonate (CS₂CO₃) in dimethyl formamide at 110° C. for 12 hours to obtain an intermediate 5. The intermediate 5 is reacted with 1.5 equiv. of chloroacetyl chloride in presence of 3 equiv. sodium bicarbonate (NaHCO₃) in dichloromethane at room temperature for 6 hours to obtain one of the 2-aminobenzophenone derivatives of Formula I consisting of N-(2-benzoylphenyl)-2-chloro-N-isopropylacetamide (IITK3028).

In another embodiment of the present invention, the process comprises reacting 2-amino-benzophenone 1 with alkyl halide (R₆—X) in presence of 3 equiv. potassium carbonate (K₂CO₃) in dimethyl formamide at 90° C. for 12 hours to obtain the intermediate 2-5.

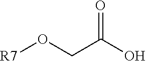

is reacted with 1.5 equiv. thionyl chloride in dichloromethane at 0° C. to room temperature for 2 hours to obtain

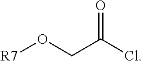

R₇ is selected from a group consisting one of phenyl, 4-fluorophenyl, 4-bromophenyl, 4-tert-butylphenyl, and pentaflurophenyl. The intermediate 2-5 is reacted with the intermediate

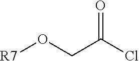

in presence of 3 equiv. sodium bicarbonate (NaHCO₃) in dichloromethane at 0° C. to room temperature for 6 hours to obtain one of the 2-aminobenzophenone derivatives of Formula I consisting one of N-(2-benzoylphenyl)-2-(4-fluorophenoxy)-N-methylacetamide (IITK3006), N-(2-benzoylphenyl)-N-methyl-2-(perfluorophenoxy)acetamide (IITK3007), N-(2-benzoylphenyl)-2-(4-(tert-butyl)phenoxy)-N-methylacetamide (IITK3008), N-(2-benzoylphenyl)-2-(4-bromophenoxy)-N-methylacetamide (IITK3009), N-(2-benzoylphenyl)-2-phenoxyacetamide (IITK3014), N-allyl-N-(2-benzoylphenyl)-2-phenoxyacetamide (IITK3020), N-allyl-N-(2-benzoylphenyl)-2-(4-fluorophenoxy)acetamide (IITK3021), N-allyl-N-(2-benzoylphenyl)-2-(perfluorophenoxy)acetamide (IITK3022), N-allyl-N-(2-benzoylphenyl)-2-(4-(tert-butyl)phenoxy)acetamide (IITK3023), N-allyl-N-(2-benzoylphenyl)-2-(4-bromophenoxy)acetamide (IITK3024), N-allyl-N-(2-benzoylphenyl)-2-(4-nitrophenoxy)acetamide (IITK3025), N-(2-benzoylphenyl)-2-phenoxy-N-(prop-2-yn-1-yl)acetamide (IITK3029), N-(2-benzoylphenyl)-2-(4-fluorophenoxy)-N-(prop-2-yn-1-yl)acetamide (IITK3030), N-(2-benzoylphenyl)-2-(perfluorophenoxy)-N-(prop-2-yn-1-yl)acetamide (IITK3031), N-(2-benzoylphenyl)-2-(4-(tert-butyl)phenoxy)-N-(prop-2-yn-1-yl)acetamide (IITK3032), and N-(2-benzoylphenyl)-2-(4-bromophenoxy)-N-(prop-2-yn-1-yl)acetamide (IITK3033).

In another embodiment of the present invention, the process comprises reacting 2-amino-benzophenone 1 with 1.5 equiv. methyl iodide in presence of 3 equiv. sodium bicarbonate (NaHCO₃) in dimethyl formamide at 110° C. for 12 hours to obtain the intermediate 2. The intermediate 2 is reacted with 1.5 equiv. electrophiles in presence of 3 equiv. base in dichloromethane at room temperature for 6 hours to obtain one of the 2-aminobenzophenone derivatives of Formula I consisting one of N-(2-benzoylphenyl)-N-methylacrylamide (IITK3010), and N-(2-benzoylphenyl)-N-methylethenesulfonamide (IITK3011).

In another embodiment of the present invention, the process comprises reacting (2-amino-5-nitrophenyl)(phenyl)methanone 6 with 1.5 equiv. methyl iodide in presence of 3 equiv. caesium carbonate (CS₂CO₃) in dimethyl formamide at 110° C. for 22 hours to obtain the intermediate 2-(methylamino)-5-nitrophenyl)(phenyl)methanone 7. The intermediate 2-(methylamino)-5-nitrophenyl)(phenyl) methanone 7 is reacted with 0.1 equiv. palladium/carbon (Pd/C) in tetrahydrofuran for 3 hours at room temperature to obtain an intermediate (5-amino-2-(methylamino)phenyl)(phenyl)methanone 9. The intermediate (5-amino-2-(methylamino)phenyl)(phenyl) methanone 9 is reacted with 1 equiv. Propargyloxycarbonyl-Cl (Poc-Cl) and 3 equiv. sodium bicarbonate (NaHCO₃) in tetrahydrofuran at room temperature for 3 hours to obtain an intermediate prop-2-yn-1-yl (3-benzoyl-4-(methylamino)phenyl)carbamate 10. The intermediate prop-2-yn-1-yl (3-benzoyl-4-(methylamino)phenyl)carbamate 10 is reacted with 2 equiv. triethyl amine and 1.5 equiv. electrophile in dichloromethane at room temperature for 6 hours to obtain one of the 2-aminobenzophenone derivatives of Formula I consisting one of prop-2-yn-1-yl (3-benzoyl-4-(2-chloro-N-methylacetamido) phenyl)carbamate (IITK3052), and prop-2-yn-1-yl (3-benzoyl-4-(2-bromo-N-methylacetamido) phenyl)carbamate (IITK3053). The electrophile is selected from a group consisting one of

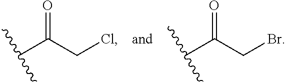 [SMILES: *C(=O)CCl ; *C(=O)CBr]

In another embodiment of the present invention, the process comprises reacting (2-aminophenyl)(4-bromophenyl)methanone with 1.5 equiv. methyl iodide in presence of 3 equiv. potassium carbonate (K₂CO₃) in dimethyl formamide at 90° C. for 12 hours to obtain an intermediate (4-bromophenyl)(2-(methylamino)phenyl)methanone. The intermediate (4-bromophenyl)(2-(methylamino)phenyl) methanone is reacted with 1.5 equiv. phenylboronic acid, 0.01 equiv. Palladium(II) acetate (Pd(OAc)₂) and 2 equiv. potassium carbonate (K₂CO₃) in dimethyl formamide at 80° C. for 4-6 hours to obtain compounds with formula

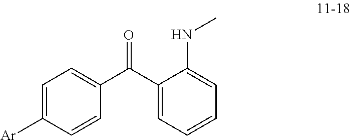

wherein,
Ar is selected from the group consisting one of

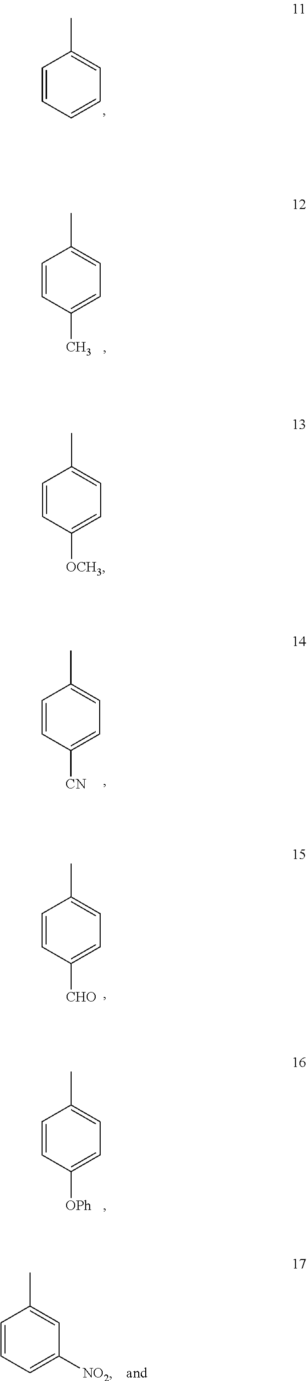

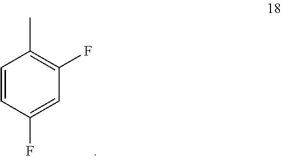

In another embodiment of the present invention, the process comprises reacting

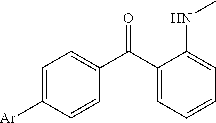

with 1.5 equiv. electrophile and 3 equiv. base in dichloromethane at room temperature for 6 hours to obtain compounds with formula

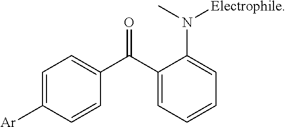

The electrophile is selected from a group consisting one of

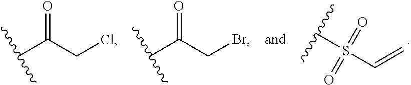

The compounds with the formula

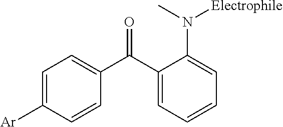

is selected from a group consisting one of N-(2-([1,1′-biphenyl]-4-carbonyl)phenyl)-2-chloro-N-methylacetamide (IITK3055), N-(2-([1,1′-biphenyl]-4-carbonyl)phenyl)-2-bromo-N-methylacetamide (IITK3056), 2-bromo-N-methyl-N-(2-(4′-methyl-[1,1′-biphenyl]-4-carbonyl)phenyl)acetamide (IITK3057), 2-chloro-N-(2-(4′-formyl-[1,1′-biphenyl]-4-carbonyl)phenyl)-N-methylacetamide (IITK3058), 2-bromo-N-(2-(4′-formyl-[1,1′-biphenyl]-4-carbonyl)phenyl)-N-methylacetamide (IITK3059), 2-chloro-N-(2-(4′-cyano-[1,1′-biphenyl]-4-carbonyl)phenyl)-N-methylacetamide (IITK3060), 2-bromo-N-(2-(4′-cyano-[1,1′-biphenyl]-4-carbonyl)phenyl)-N-methylacetamide (IITK3061), 2-chloro-N-methyl-N-(2-(3′-phenoxy-[1,1′-biphenyl]-4-carbonyl)phenyl)acetamide (IITK3062), 2-bromo-N-methyl-N-(2-(3′-phenoxy-[1,1′-biphenyl]-4-carbonyl)phenyl)acetamide (IITK3063), 2-chloro-N-(2-(4′-methoxy-[1,1′-biphenyl]-4-carbonyl)phenyl)-N-methylacetamide (IITK3064), 2-bromo-N-(2-(4′-methoxy-[1,1′-biphenyl]-4-carbonyl)phenyl)-N-methylacetamide (IITK3065), 2-chloro-N-(2-(2′,4′-difluoro-[1,1′-biphenyl]-4-carbonyl) phenyl)-N-methylacetamide (IITK3066), 2-bromo-N-(2-(2′,4′-difluoro-[1,1′-biphenyl]-4-carbonyl)phenyl)-N-methylacetamide (IITK3067), 2-chloro-N-methyl-N-(2-(3′-nitro-[1,1′-biphenyl]-4-carbonyl)phenyl) acetamide (IITK3068), 2-bromo-N-methyl-N-(2-(3′-nitro-[1,1′-biphenyl]-4-carbonyl)phenyl)acetamide (IITK3069), N-(2-([1,1′-biphenyl]-4-carbonyl)phenyl)-N-methylethenesulfonamide (IITK3070), N-(2-([1,1′-biphenyl]-4-carbonyl)phenyl)-2-bromo-N-(prop-2-yn-1-yl)acetamide (IITK3071), and N-(2-([1,1′-biphenyl]-4-carbonyl)phenyl)-2-chloro-N-(prop-2-yn-1-yl)acetamide (IITK3072).

In another embodiment of the present invention, the process comprises reacting (2-amino-5-nitrophenyl)(phenyl)methanone 6 with 1.5 equiv. methyl iodide in presence of 3 equiv. cesium carbonate (CS₂CO₃) in dimethyl formamide at 110° C. for 12 hours to obtain an intermediate 2-(methylamino)-5-nitrophenyl)(phenyl)methanone 7. The intermediate 2-(methylamino)-5-nitrophenyl)(phenyl) methanone 7 is reacted with 1.5 equiv. of chloroacetyl chloride in presence of 3 equiv. sodium bicarbonate (NaHCO₃) carbonate in dichloromethane at room temperature for 6 hours to obtain N-(2-benzoyl-4-nitrophenyl)-2-chloro-N-methylacetamide (IITK3073).

The present invention is explained further in the following specific examples, which are only by way of illustration and are not to be construed as limiting the scope of the invention.

The present invention provides a screening of covalent small molecules library for identification of potent ferroptosis inducers in cancer cells is provided. Thiol-reactive electrophilic small molecules chloro-(IITK3001) and bromoacetamides (IITK3002) are identified as effective inhibitors of cancer cell proliferation at nanomolar concentrations (EC₅₀=70 nM-IITK3001) and 3 nM-IITK3002). The mechanism of cell death induction is established which is ferroptosis precisely for lead molecules and no other pathways like apoptosis or necroptosis are in operation. The subsequent in-gel fluorescence analysis of cellular proteome treated with lead small molecules is identified as a single protein being covalently modified. Further, we have demonstrated that our molecules are capable of alkylating the thiols of cysteines indicating the modification of catalytic cysteine residues in the target protein to inhibit the function of the protein. Together, we identified a unique class of haloacetmaides as covalent modifiers of cysteine-containing proteins that is linked to ferroptosis in cancer cells.

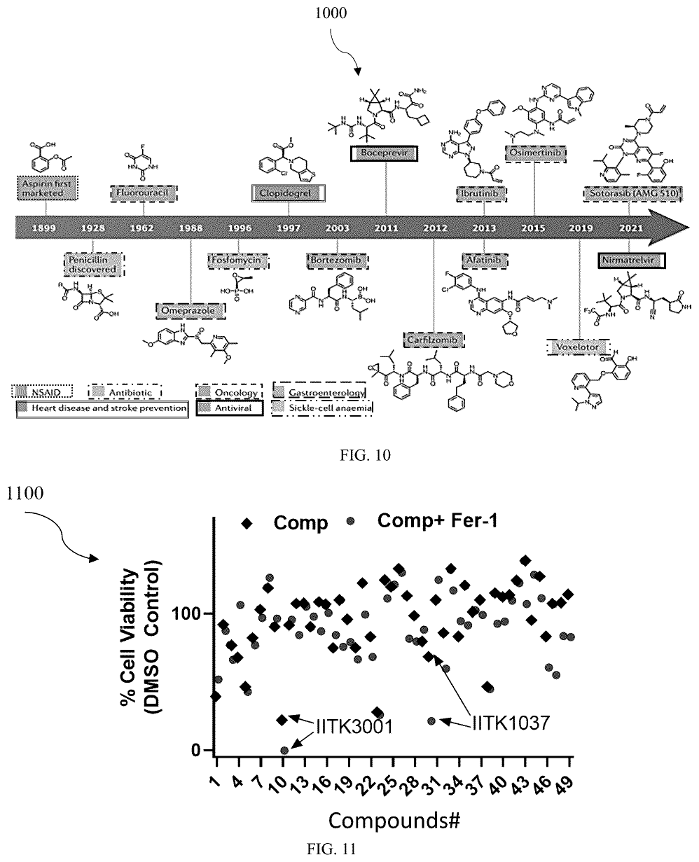

FIG. 10 depicts major covalent drug development timeline as disclosed in a prior art Boike et. al., 2022.

A small molecule is utilized as a screening platform for the identification of new ferroptosis inducers by exposing cells to our small molecule library in the presence and absence of ferrostatin-1 (Fer-1, 10 μM). The molecular hits capable of inhibiting the cancer cells proliferation (hepatocellular carcinoma, Huh-7 cells) at 5 μM dose and a reversed effect in the presence of Fer-1 were chosen for further mechanistic evaluation and structure-activity-relationship analysis. We have identified a single-digit nanomolar inhibitor of Huh-7 cells (IITK-3001; EC₅₀: 3 nM), the most potent amongst the ferroptosis inducers known in the literature. Our effort to identify the molecular target of IITK3001 using activity-based protein profiling and in-gel fluorescence analysis identified a unique protein target with a molecular weight range of ˜55 kDa, completely different from thus far known ferroptosis-linked protein targets. The process of chemical proteomics to uncover the identity of the protein target is been explored.

### Results and Discussions

FIG. 11 depicts screening of electrophilic small molecules has identified IITK3001 and IITK1037 as potent inhibitors of Huh-7 cells, in accordance with an embodiment of the present invention. The viability of Huh-7 cells was recorded using a luminescence-based CellTiter-Glo® assay. The screening concentration of the molecules were 10 μM with or without Fer-1 (10 μM) in DMEM media (>0.1% DMSO as control).

### Screening of Electrophiles Library for the Identification of Ferroptosis Inducers

Ferroptosis is a unique form of cell death mechanism mediated by the accumulation of lipid peroxides in cells which is distinct from healthy cells' apoptosis, a programmed senescence route. We aimed to exploit the induction of ferroptosis in cancer cells using covalent small molecules as a potential strategy for cancer therapeutic development. The involvement of redox proteins possessing cysteine in the active sites could play a major role in suppressing ferroptosis Therefore, we designed small molecule libraries with diverse chemical scaffolds coupled with thiol-reactive electrophilic warheads (including haloacetamides, acrylamides, chalcones, vinyl sulfones and maleimides) as a design for developing ferroptosis-inducing covalent therapeutics. We used various amide bond formation chemistry to construct the library members and these molecules are well characterized by various spectroscopic and mass spectrometry techniques. Once confirmed, these thiol reactive library members were screened in a cancer cell viability assay in combination with ferrostatin-1 (Fer-1), a ferroptosis inhibitor as depicted in FIG. 11. The lead ferroptosis inducers identified in this experiment would exhibit a suppressed potency to induce antiproliferative activity only when combined with Fer-1 as depicted in FIG. 11. We have screened a total of ˜50 electrophilic small molecules to prevent the proliferation of hepatocellular carcinoma (Huh-7 cells) in combination with and without Fer-1. From our initial screening, we identified haloacetamides of 2-aminoalkylbenzophenone as potential ferroptosis inducers as depicted in FIG. 11.

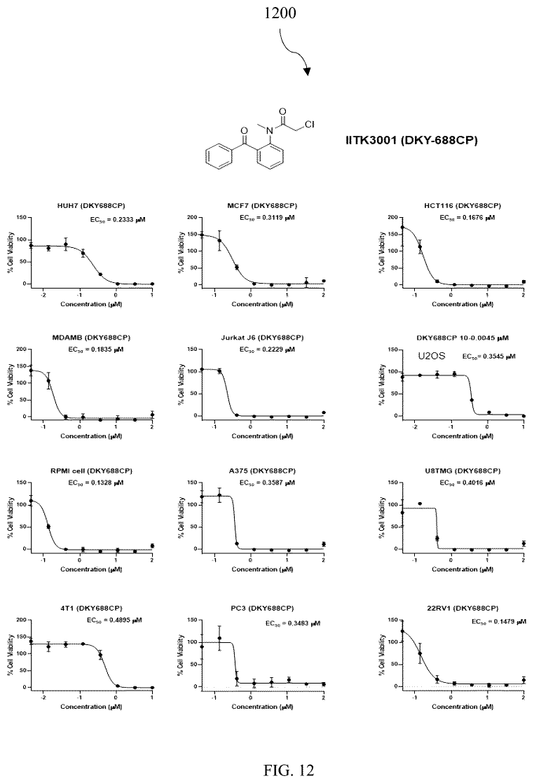

FIG. 12 depicts IITK3001 dose-dependently inhibited the proliferation of twelve-different cancer cell lines, in accordance with an embodiment of the present invention. The viability of screened cancer cells was recorded using a luminescence-based CellTiter-Glo® assay for the ATP quantification. The screening concentration of the molecule ranged from 100/10 μM to 45/4.5 nM with an 8-point-3-fold dilution series in DMEM media (>0.1% DMSO as control). Each experiment was conducted in more than two biological replicates with three technical replicates. The error bar is a representation of standard deviation among the technical replicates.

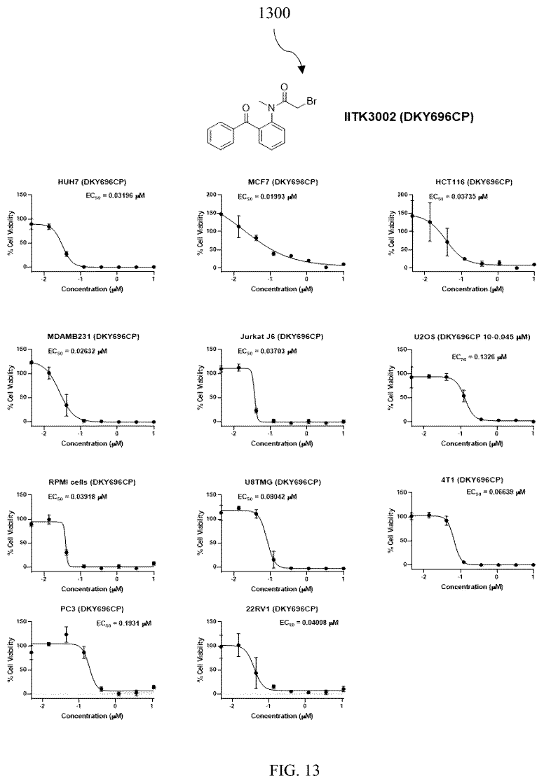

FIG. 13 depicts IITK3002 dose-dependently inhibited the proliferation of twelve-different cancer cell lines, in accordance with an embodiment of the present invention. The viability of screened cancer cells was recorded using a luminescence-based CellTiter-Glo® assay for the ATP quantification. The screening concentration of IITK3002 ranged from 10 μM to 4.5 nM with an 8-point-3-fold dilution series in DMEM media (>0.1% DMSO as control). Each experiment was conducted in more than two biological replicates with three technical replicates. The error bar is a representation of standard deviation among the technical replicates.

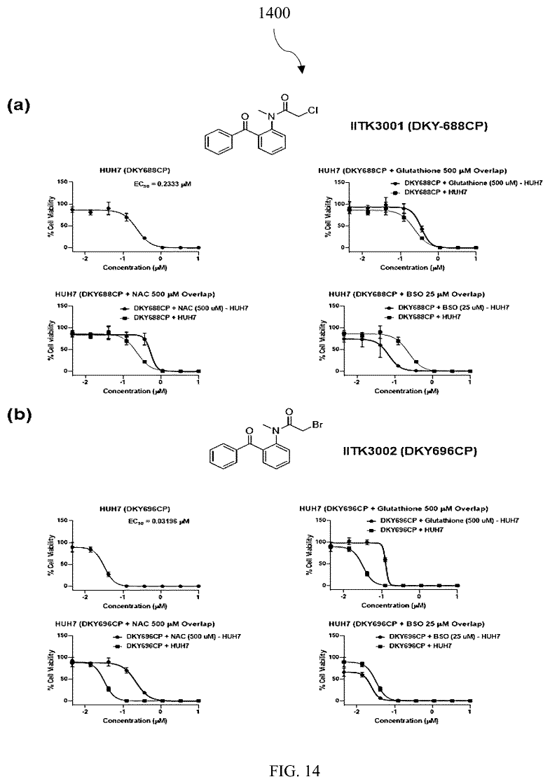

FIG. 14 depicts a combination experiment indicates the involvement of thiol modulation by IITK3001/IITK3002 on the viability of Huh-7 and 4T1 cells, in accordance with an embodiment of the present invention. The viability of screened cancer cells was recorded using a luminescence-based CellTiter-Glo® assay for the ATP quantification. The screening concentration of (a) IITK3001 and (b) IITK3002 ranged from 10 uM to 4.5 nM with an 8-point-3-fold dilution series in DMEM medium (>0.1% DMSO as control). For combination experiments, 500 μM of NAC or glutathione and 25 μM of BSO were used. Each experiment was conducted in more than two biological replicates with three technical replicates. The error bar is a representation of standard deviation among the technical replicates.

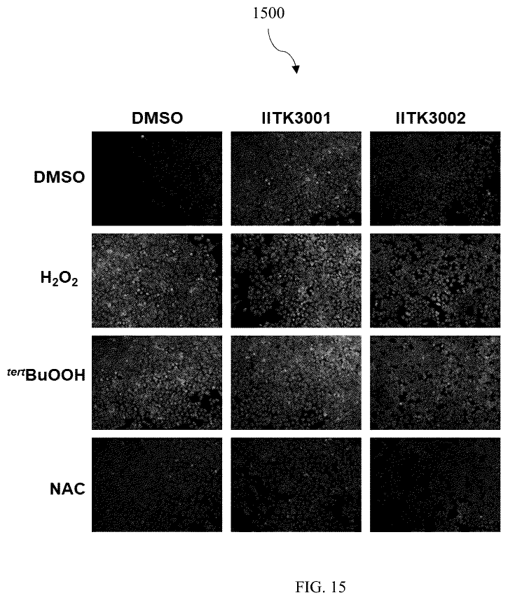

FIG. 15 depicts an intracellular ROS generation by IITK3001/IITK3002 and its quenching by the combination of NAC captured using H2-DCFDA oxidation assay in Huh-7 cells, in accordance with an embodiment of the present invention. We used fluorescence imaging-based oxidation of H₂-DCFDA by ROS for assessing intracellular oxidative stress. Huh-7 cells were treated at 37° C. for 30 min with IITK3001 and IITK3002 (20 μM), H₂O₂ (100 μM), ^{tert}BuOOH (20 μM), NAC (0.5 mM) and 0.1% DMSO as control. Each experiment was conducted in more than two biological replicates with three technical replicates.

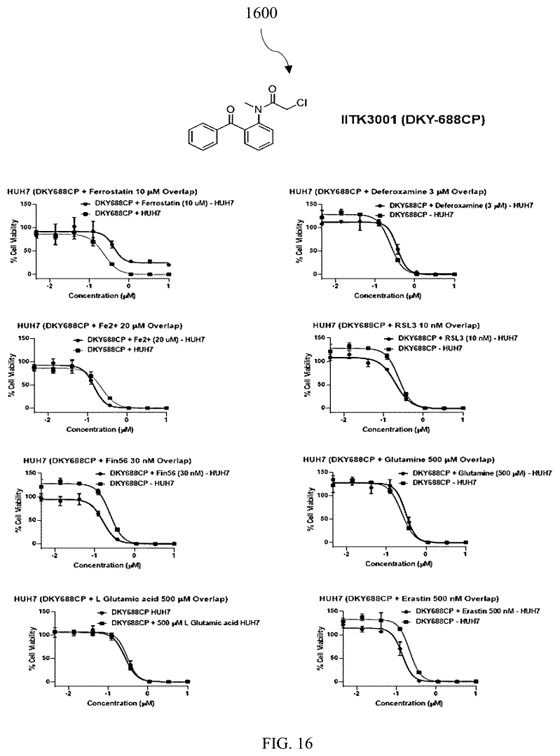

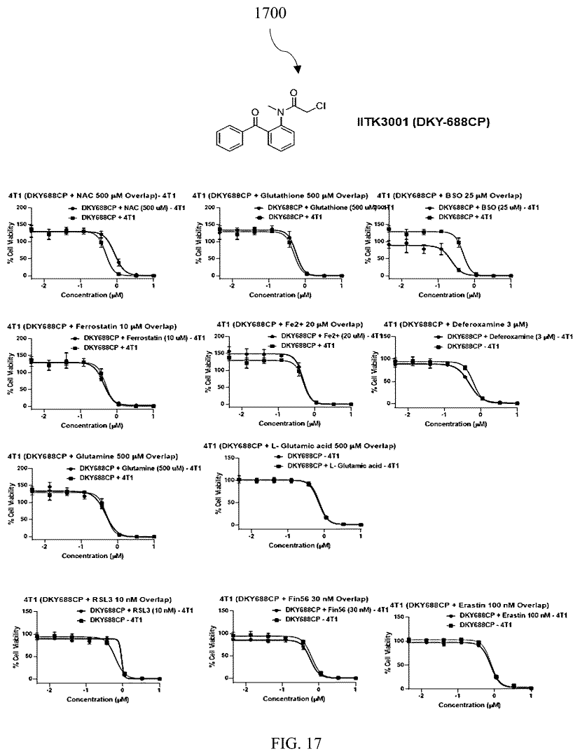

FIG. 16 depicts ferroptosis induction as a cell death mechanism is established for IITK3001 using combination experiments with ferroptosis inducers and inhibitors, in accordance with an embodiment of the present invention. The screening concentration of (a) IITK3001 and (b) IITK3002 ranged from 10 μM to 4.5 nM with an 8-point-3-fold dilution series in DMEM medium (>0.1% DMSO as control). For combination experiments, Ferrostain-1 (10 μM),Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation among the technical replicates.

FIG. 17 depicts IITK3001-mediated ferroptosis induction as a cell death mechanism in 4T1 cells is recorded, in accordance with an embodiment of the present invention. The screening concentration of (a) IITK3001 and (b) IITK3002 ranged from 10 μM to 4.5 nM with an 8-point-3-fold dilution series. For combination experiments, Ferrostain-1 (10 μM),Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation among the technical replicates.

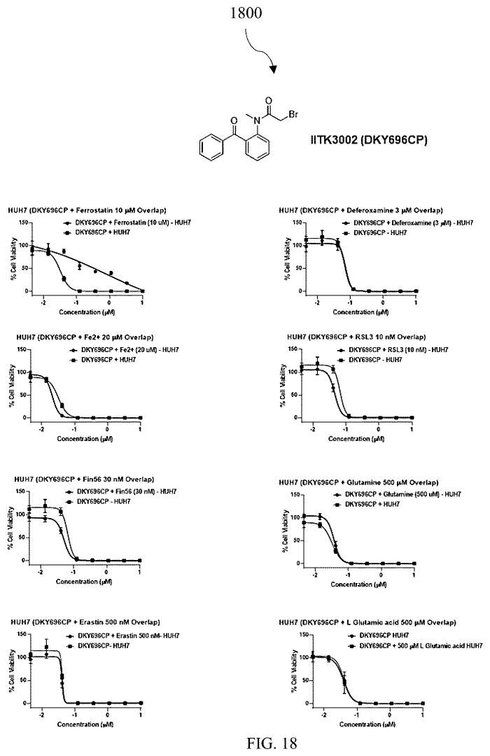

FIG. 18 depicts ferroptosis induction as a cell death mechanism is established for IITK3002 using combination experiments with ferroptosis inducers and inhibitors, in accordance with an embodiment of the present invention. The screening concentration of IITK3002 ranged from 10 μM to 4.5 nM with an 8-point-3-fold dilution series in DMEM medium (>0.1% DMSO as control). For combination experiments, Ferrostain-1 (10 μM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation among the technical replicates.

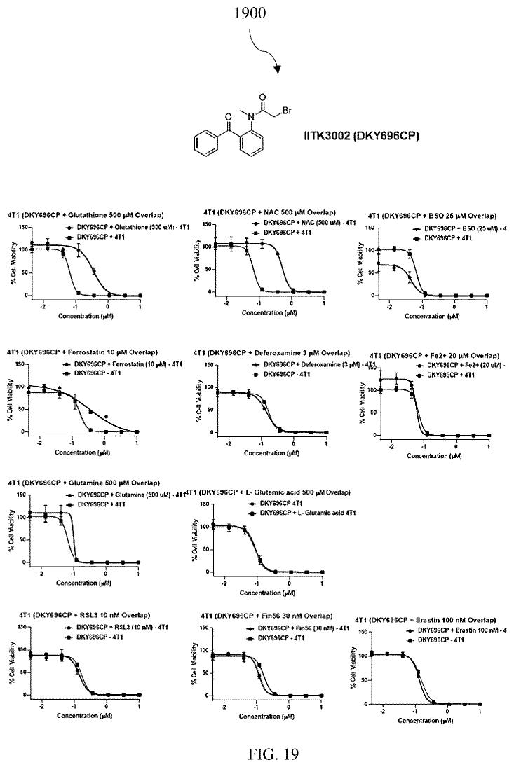

FIG. 19 depicts IITK3001-mediated ferroptosis induction as a cell death mechanism in 4T1 cells is recorded, in accordance with an embodiment of the present invention. The screening concentration of (a) IITK3001 and (b) IITK3002 ranged from 10 μM to 4.5 nM with an 8-point-3-fold dilution series. For combination experiments, Ferrostain-1 (10 μM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation among the technical replicates.

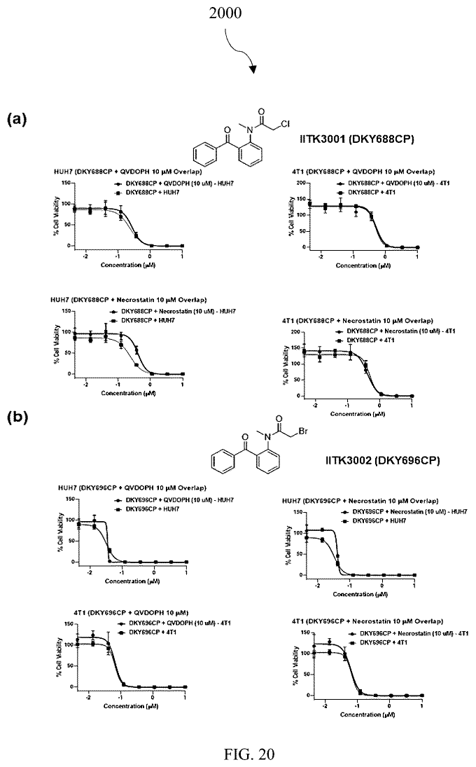

FIG. 20 depicts combining apoptosis and necroptosis inhibitors ruled out the possibility of respective cell death mechanisms upon treatment with IITK3001 or IITK3002 in Huh-7 and 4T1 cells, in accordance with an embodiment of the present invention. The screening concentration of IITK3001/IITK3002 ranged from 10 μM to 4.5 nM with an 8-point-3-fold dilution series in DMEM medium (>0.1% DMSO as control). For combination experiments, QVD-OPh and Necrsotatin-1 (10 μM) were used. Each experiment was conducted in more than two biological replicates with three technical replicates. The error bar is a representation of standard deviation among the technical replicates.

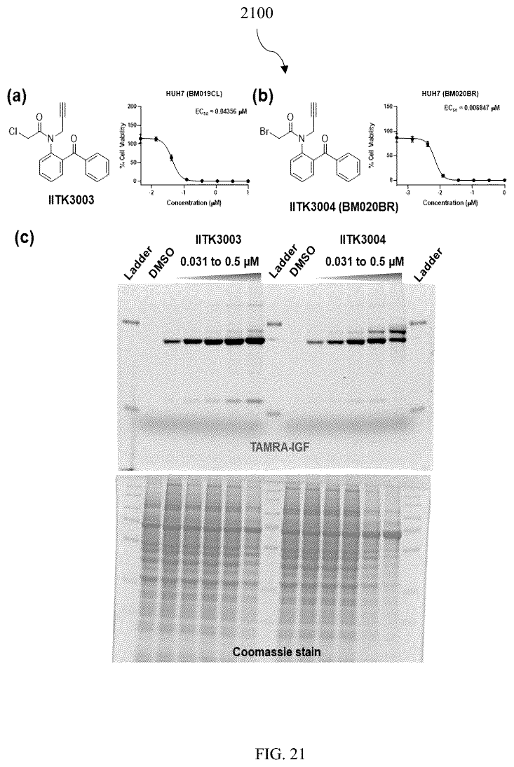

FIG. 21 depicts cell viability assessment of alkyne-versions (IITK3003 and IITK3004) confirmed retaining the potency in Huh-7 cells and in-gel fluorescence analysis revealed a single major band indicating a selective protein modification, in accordance with an embodiment of the present invention. (a) For the cell viability analysis, the concentration of the molecules (a) IITK3003 and (b) IITK3004 used were ranging from 10 μM to 4.5 nM with an 8-point-3-fold dilution series in DMEM medium (>0.1% DMSO as control). (c) For combination experiments, QVD-OPh and Necrsotatin-1 (10 μM) were used. Each experiment was conducted in more than two biological replicates with three technical replicates. The error bar is a representation of standard deviation among the technical replicates.

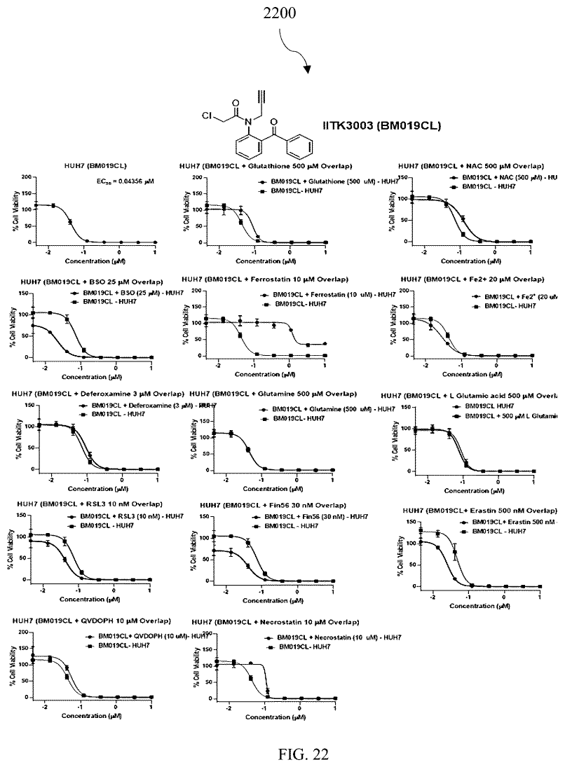

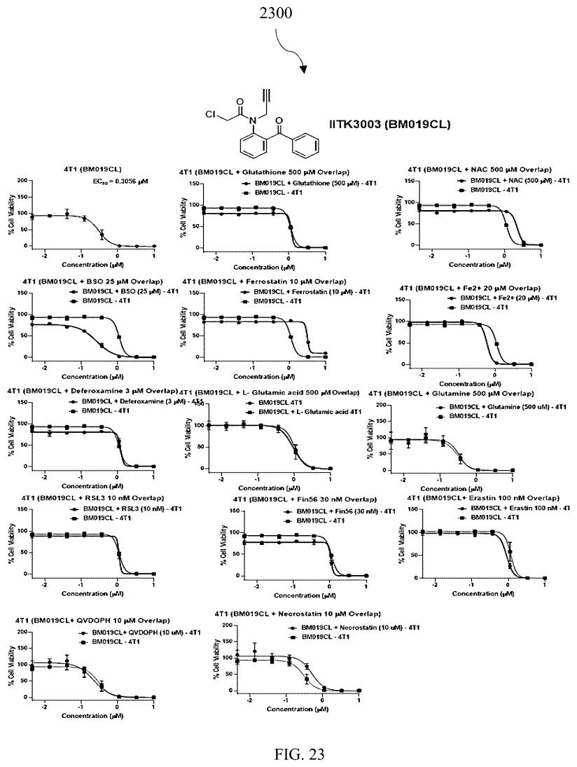

FIG. 22 depicts ferroptosis induction was established for IITK3003 using combination experiments in Huh-7 cells, in accordance with an embodiment of the present invention. The screening concentration of IITK3002 ranged from 10 uM to 4.5 nM with an 8-point-3-fold dilution series in DMEM medium (>0.1% DMSO as control). For combination experiments, Ferrostain-1 (10 PM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation among the technical replicates.

FIG. 23 depicts ferroptosis induction is established for IITK3003 using combination experiments in 4T1 cells, in accordance with an embodiment of the present invention. The screening concentration of the molecules (a) IITK3001 and (b) IITK3002 ranged from 10 μM to 4.5 nM with an 8-point-3-fold dilution series. For combination experiments, Ferrostain-1 (10 μM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation between the technical replicates.

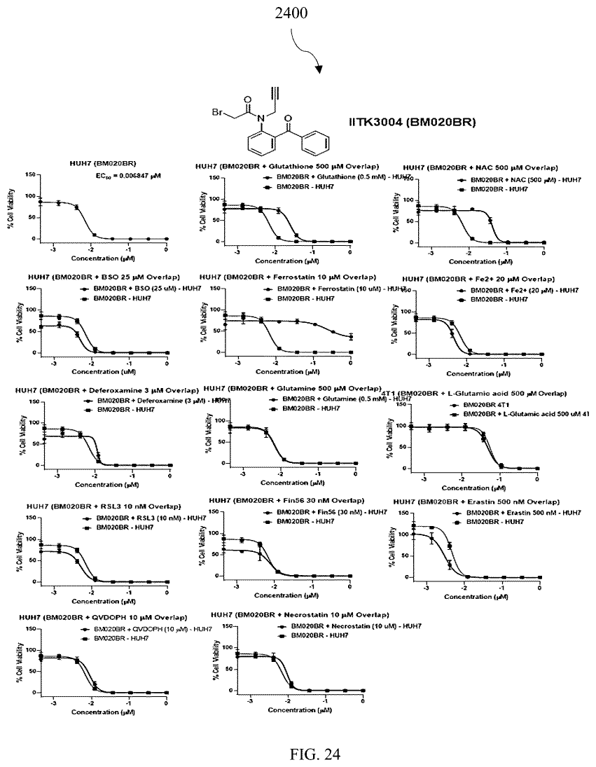

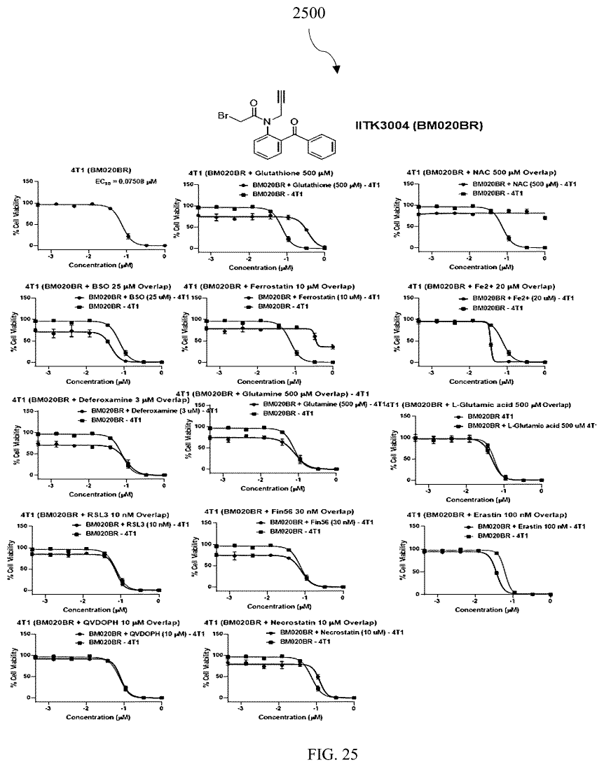

FIG. 24 depicts ferroptosis induction was established for IITK3004 using combination experiments in Huh-7 cells, in accordance with an embodiment of the present invention. The screening concentration of the molecules (a) IITK3001 and (b) IITK3002 ranged from 10 μM to 4.5 nM with a 8-point-3-fold dilution series. For combination experiments, Ferrostain-1 (10 PM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation between the technical replicates.

FIG. 25 depicts ferroptosis induction is established for IITK3004 using combination experiments in 4T1 cells, in accordance with an embodiment of the present invention. The screening concentration of the molecules (a) IITK3001 and (b) IITK3002 ranged from 10 μM to 4.5 nM with an 8-point-3-fold dilution series. For combination experiments, Ferrostain-1 (10 μM), Deferoxamine (3 μM), RSL3 (10 nM), FIN56 (30 nM), glutamine (0.5 mM), glutamic acid (0.5 mM), Erastin (0.5 μM) were used. Each experiment was conducted in more than two biological replicates with three technical replicates. The error bar is a representation of standard deviation between the technical replicates.

### Establishing the Ferroptosis Mechanism of Hit Molecules:

a. Dose-Dependent Effect Validation in Cancer Cells:
We then evaluated in these molecules an 8-point-3-fold dilution dose series starting from 100 μM to derive the molecules' effective concentration to exhibit antiproliferative activity. We identified N-methylchloroacetamide (IITK3001) as the most potent inhibitor of cancer cell proliferation with an EC50 of 120 nM in Huh-7 cells as depicted in FIG. 12. In parallel, we have also synthesized the bromoacetamide (IITK3002) version of the lead molecules which is slightly more reactive with thiol nucleophiles when compared to chlorooacetamide warheads as depicted in FIG. 13. IITK3002 possessed antiproliferative activity with an EC₅₀ of 30 nM against in Huh-7 cells, which is not surprising as chloroacetamide is a weaker electrophile than the bromoacetamide version as depicted in FIG. 12, and FIG. 13. Haloacetamides of alkylated-2-aminobenzophenones showed strong antiproliferative properties at nanomolar concentrations and were carried forward for further mechanistic evaluations. In addition, we also evaluated the molecules' antiproliferative potency against a panel of twelve different cancer phenotypes. The set of cancer cell lines included liver, breast, colon, myeloma, bone, blood, skin, brain and prostate lineages (FIG. 2, 3, Table 1). We have observed a strong inhibitory effect at nanomolar EC₅₀ concentration ranges (for IITK3001: 132 to 490 nM & IITK3002: 20-132 nM) as depicted in FIG. 12, FIG. 13 and Table 1.

TABLE 1
Cell viability data of IITK3001 against a panel of 12 cancer cells:

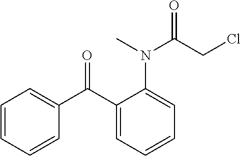 [SMILES: CN(C(=O)CCl)c1ccccc1C(=O)c1ccccc1]

IITK 3001

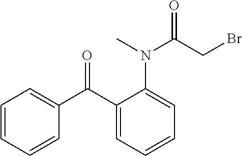 [SMILES: CN(C(=O)CBr)c1ccccc1C(=O)c1ccccc1]

IITK 3002

| S. No | Cell Lines | IITK3001 EC₅₀ (μM) | IITK3002 EC₅₀ (μM |
|---|---|---|---|
| 1 | Huh-7 | 0.233 | 0.032 |
| 2 | MCF7 | 0.312 | 0.020 |
| 3 | HCT116 | 0.168 | 0.037 |
| 4 | MDA-MB-231 | 0.184 | 0.026 |
| 5 | Jurkat J6 | 0.222 | 0.037 |
| 6 | U2OS | 0.355 | 0.133 |
| 7 | RPMI 8226 | 0.133 | 0.039 |
| 8 | A375 | 0.352 | N.D. |
| 9 | U87MG | 0.402 | 0.080 |
| 10 | 4T1 | 0.499 | 0.066 |
| 11 | PC3 | 0.348 | 0.193 |
| 12 | 22Rv1 | 0.148 | 0.040 |

N.D. = Not determined

b. Effect of Thiols and Ferroptosis Modulators on the Hit Molecules' Antiproliferative Potency:
Ferroptosis is directly controlled by the intracellular thiol concentrations in the form of cysteine and glutathione. Several oxidative mechanisms are well-wired in ferroptotic cell death and we set out to investigate the precise molecular mechanism of our lead molecules. Both cysteine and glutathione (GSH) are the enriched thiol-containing small molecule antioxidant system that are essential regulators of redox modulation in cells including ferroptosis. Further, the enzyme glutathione peroxidase 4 (GPX4) is well characterized to specifically quench the lipid peroxide equivalents in cells through the conversion of oxidized glutathione dimer into reduced glutathione in cells thereby restoring the reduced environment in cells. Therefore, first, we assessed the ability of N-acetylcysteine (NAC) and GSH to suppress our lead molecules antiproliferative activity (FIG. 4a, b). We found a drastic reduction in the molecules' potency with IITK3001 (>1000-fold reduction) and IITK3002 (>500-fold reduction) (FIG. 14a, b). Supporting that, the addition of DL-Buthionine sulfoximine (BSO), an inhibitor of y-glutamylcysteine synthetase (glutathione biosynthesis) combination with our lead molecules potentiated the overall inhibitory capacity (FIG. 14a, b). Transportation of cysteine inside the cells is essential for the biosynthesis of glutathione, which is executed by the System X_{c}⁻ antiporter assembly at the cellular plasma membrane. We investigated whether the lead molecules might inhibit the antiporter system to promote lipid peroxide accumulation inside the cells and translate into ferroptosis. Therefore, we examined the effect of our small molecules in combination with a known inhibitor of System X_{c}⁻ antiporter, erastin to visualize any additive effect at a relatively non-toxic concentration (2-fold less than the EC₅₀ dosage, as depicted in FIG. 16-19. We found no effect of erastin addition on the antiproliferative activity of both IITK3001 and IITK3002 and thereby ruled out the possibility of antiporter inhibition as the mechanism of action as depicted in FIG. 16-19.
Next, the accumulation of iron is essential for ferroptosis to be initiated, we used a standard iron chelator, deferoxamine to investigate the effect of iron depletion on our molecules' potency. Identical to erastin, we did not observe any significant effect on the lead molecules' potency, suggesting the molecule effect is not the upstream where the iron accumulation is essential. Finally, we assessed the effect of Ferrostain-1 (Fer-1) and Liproxstatin-1 (Lip-1), antioxidants that function by trapping reactive radicals in cells, particularly lipid-peroxide radicals, and preventing the accumulation of lipid peroxides in cells. We found an excellent correlation that a more than 500-fold decrease in the potency of our lead molecules to prevent cancer cell proliferation by both Fer-1 and Lip-1 as depicted in FIG. 16-19. Whereas the addition of non-toxic dose of other GPX4-inhibition-driven ferroptosis inducers, RSL3 and FIN56 did not affect the dose curve of the lead molecule, perhaps, the molecular target is not GPX4 as depicted in FIG. 16-19. The accumulation of lipid peroxides and overall ROS was also detected using a fluorescence-based H₂DCF-DA assay as depicted in FIG. 15. We could see a drastic enhancement in the fluorescence intensity upon exposure to Hydrogen peroxide and our lead molecules as depicted in FIG. 15. The addition of NAC was found to suppress the same extent, the overall fluorescence increase, thereby confirming the accumulation of intracellular ROS upon treatment with our lead molecules as depicted in FIG. 15. These data strongly suggested that the cell death mechanism induced by lead molecules is ferroptosis upon treatment with IITK3001/IITK3002. To eliminate other cell death pathways like apoptosis or necrosis being initiated upon our small molecules' treatment, we combined inhibitors of apoptosis (QVD-OPh) and necrosis (Necrostatin-1) with IITK3001/IITK3002 and observed no apparent deviation in the overall antiproliferative activity as depicted in FIG. 20a, b. All of the described experiments were repeated in another cancer cell line, 4T1 (a breast cancer phenotype) and we observed a similar trend with all the combinatorial evaluations, except for IITK3001 with Fer-1 (the effect was negligible, as depicted in FIG. 20a, b. Together, these combination cell viability experiments highlighted a highly regulated ferroptosis induction in cancer cells by IITK3001/IITK3002 for their mechanism of action.

### Target Identification:

Our next goal was to track the molecular target(s) of the hit molecules in hepatocellular carcinoma cells. Our molecules possess an electrophilic warhead which is expected to offer a covalent modification to the biological targets. Then we translated the hit molecules into alkyne containing version by replacing the methyl group with a propargyl-functional group. This alkyne handle enables us to perform the activity-based protein profiling (ABPP) through click chemistry with a fluorescent azide (in-gel fluorescence analysis) or biotin-tag (pulldown-mass spectrometry) for the target identification. Initially, after synthesis and characterization of the alkyne versions IITK3003 (of IITK3001) and IITK3004 (of IITK3002), we assessed the molecules' potency in Huh-7 cells before performing ABPP experiments. We found the bromoacetamide derivative (IITK-3004) was the best molecule with EC₅₀ of 7 nM potency in Huh-7 cells and the chloroacetamide-derivative IITK3003 exhibited an EC₅₀ of 44 nM (FIG. 11a, b). Therefore, the potency of the molecules if not affected, has slightly improved upon the replacement of methyl group with an alkyne tag. We used these molecules to perform in-gel fluorescence (IGF) analysis experiment, where we exposed Huh-7 cells to our molecules for 6 h and isolated the proteome for a Cu(I)-catalyzed azide-alkyne click reaction with a fluorescence azide (TAMRA-N₃) in PBS. Then, the proteome was resolved in an SDS-Page and visualized using a fluorescence gel scanner as depicted in FIG. 21c. To our surprise, we observed just a one single band around 40-50 kDa appearing in the IGF, indicative this could be reacting with one single protein selectively in the cellular proteome as depicted in FIG. 21c. We have performed the experiment with varied dose to capture the effect is not random but highly dose dependent. Next, we performed click chemistry with a biotin-azide and used streptavidin magnetic beads to pull down the modified proteins. Currently, we are following the on-bead digestion protocol to digest the protein for a mass spectral analysis for the identification of target protein (s). Then we will recombinantly express the protein and perform mass spectral analysis to pinpoint the site of modification in the target proteins. If the protein is functional, we will perform their functional inhibition assay to capture their effect on the isolated protein. Finally, we will execute the pathway analysis to derive detailed link for our lead molecules mechanism of action.

### Establishing the Ferroptotic Mechanism for the Alkyne-Derivatives:

Astonishingly, the alkyne-version of lead molecules (IITK3003 and IITK3004) exhibited exceptional inhibitory potency against Huh-7 cells (60 and 3 nM respectively) and labeled a single protein majorly in the pool of cellular proteome in Huh-7 cells as depicted in FIG. 22-25. Therefore, we aimed to ensure the mechanism of action is still ferroptosis after replacing the methyl group with alkyne linkers in the lead scaffolds. We have performed all the set of experiments conducted with IITK3001 and IITK3002, like, combination with GSH, NAC, BSO, Fer-1, iron salt, deferoxamine, RSL3, FIN56, Erastin, glutamine, glutamic acid, QVD-OPh and necrostatin-1. We have observed a remarkable reproducibility with IITK3003 and IITK3004 as in the parent molecules that these are exceptionally selective ferroptosis inducers as depicted in FIG. 22-25.

### Hit to Lead Optimization:

a. Electrophilic Warhead Optimization:
Once we identified 2-benzoylphenyl-a-haloacetamide as the lead scaffold, we have investigated the structure-activity-relationship analysis to precisely capture the effect of various chemical handles of the scaffold and use the knowledge to improve the overall potency and selectivity of this molecule to prevent cancer cell proliferation. First, we changed the haloacetamide electrophilic unit with various other electrophiles with a characteristic high, moderate, and weak reactivity with thiols when compared to chloroacetamide. Therefore, we opted vinylsulfonamide (highly reactivity), acrylamide (moderate reactivity), and phenoxyacetamides (weak reactivity warheads) for incorporation in the place of haloacetamide warhead of the top hit scaffold. Once synthesized, these molecules were evaluated to assess their antiproliferative potency against Huh-7 cells (Table 2). As expected, the introduction of weakly reactive electrophilic warheads, phenoxyactamides, completely lost the activity against Huh-7 cells. Conversely, moderately reactive acrylamide was found to show weak inhibitory activity against the proliferation of Huh-7 cells than the haloacetamides. Surprisingly, vinylsulfonamide, a more reactive electrophilic warhead than chloroacetamide, showed a reduced activity like acrylamides (Table 2). Perhaps, the secondary interactions of the electrophilic warhead with the target proteins might play additional roles. Therefore, we shifted our focus to optimize the rest of the core scaffold for further SAR analysis.
b. Role of N-Substitutions:
Next, we synthesized the NH-derivative of haloacetamides to capture the importance of N-alkylation on the activity of our hit molecule. Interestingly, the absence of methyl-group on the acetamide-nitrogen of the hit molecule exhibited weakened potency to inhibit the proliferation of Huh-7 cells (Table 2). From the X-ray diffraction analysis of crystals of NH-derivative IITK3013 showed a hydrogen bonding between —NH and the carbonyl oxygen to form a 6-membered ring structure. Perhaps the hydrogen bonding induced planarity around the warhead influences the molecules' weakened potency. Conversely, the additional flexibility in the lead molecule (tertiary amide) might contribute to the strong antiproliferative activity of the hit molecule. To test this proposition, we synthesized two independent analogs, haloacetamides of 2-amino-anthraquinone and N-benzodiazepine, then we evaluated the molecules' potency in a cell viability analysis of Huh-7 cells. Indeed, both molecules showed more than a 10-fold reduction in activity when compared to IITK3001 (Table 2). Then, we introduced a methyl group on the 2-aminoanthraquinone to extract the hydrogen bonding effect from planarity. In this case, we found no significant improvement in the overall potency, therefore we concluded the avoiding both the hydrogen bonding and planarity is essential to preserve the potency of the scaffold (Table 2). To further strengthen the contribution arising from N-alkylation, we replaced the methyl group with various alkyl groups including allyl, benzyl, and propargyl functionalities on the haloacetamide hit molecules. The cell viability analysis of Huh-7 cells revealed a comparable activity for the methyl, allyl, and propargyl derivatives, while, a reduction in the potency was observed for benzyl derivatives (Table 2). Enhancing the bulkiness around the electrophilic warhead seemed to affect the potency (Table 2).
c. Importance of Benzophenone Scaffold in the Active Pharmacophore:
With our systematic approach of understanding the importance each accessible handle of the lead molecules, next we moved onto understanding the importance of the benzophenone scaffold for the activity of our lead molecules. To that end, we have synthesized and evaluated three derivations of lead molecules. First, we removed the benzoyl group and synthesized the haloacetamides of aniline. Second, we replaced the benzoyl-group ring with acetyl-group (acetophenone) and synthesized the NH- and N-Methyl derivatives of haloacetmaides. Third, we introduced an additional methylene bridge between the warhead and the aromatic ring in the form of benzyl-haloacetamides. All these derivatives invariably suffered in preventing Huh-7 cell proliferation at the comparable nanomolar, EC₅₀ concentrations (Table 2).
d. Effect of Extension on the Benzophenone Scaffold:
The lead molecules identified in our studies are relatively small with less than 300 Da molecular weight, which provides room for additional modifications to improve the potency by following within the medicinal chemistry guideline of <500 Da molecular weight. Finally, we have assessed the effect of substitutions on the benzophenone scaffold. First, we observed a reduction in the potency when we introduced an electron-withdrawing nitro group on the 4^{th} position of the electrophile-carrying aryl-ring system. Then, we reduced the nitro to amine and synthesized alkylated versions of 4-amino-benzophenones-haloacetamides. We did not observe any enhancement in the antiproliferative activity (Table 2). We also synthesized the vinylsulfonamide version of the same molecule and the antiproliferative activity was only comparable to haloacetmaides (Table 2). Therefore, any modification to the electrophile-carrying aryl ring system found loses its potency significantly. Next, we moved on to understanding the effect of substitutions on the benzoyl-ring system. We first synthesized a 4-bromobenzophenone version of haloacetamides and these molecules were characterized to preserve their potency in inhibiting Huh-7 cells. Next, we exploited the bromo-substitution in a Suzuki coupling chemistry to synthesize a small library of twelve molecules, 4-aryl-benzophenone versions and transformed them into haloacetamides (Table 2). All these molecules exhibited exceptional antiproliferative activity with less than 100 nM EC₅₀ concentrations and the best molecule was 4-phenyl-benzophenone-chloroacetamide with an EC₅₀ of 2 nM against Huh-7 cell proliferation (Table 2).

TABLE 2
Cell viability data of our library of molecules against Huh-7 cells

| S. | Compd | | EC₅₀ (μM) in various cancer cells | | | | |
|---|---|---|---|---|---|---|---|
| No | Code | Structure | Huh-7 | U2OS | A549 | PC3 | 22Rv1 |
| 1 | IITK3001 | 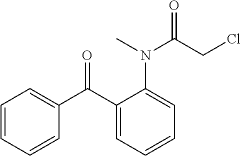 [SMILES: CN(C(=O)CCl)c1ccccc1C(=O)c1ccccc1] | 0.2333 | 0.35 | 0.61 | 0.35 | 0.15 |
| 2 | IITK3002 | 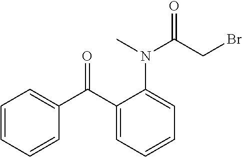 [SMILES: CN(C(=O)CBr)c1ccccc1C(=O)c1ccccc1] | 0.03196 | 0.13 | 0.03 | 0.19 | 0.04 |
| 3 | IITK3003 | 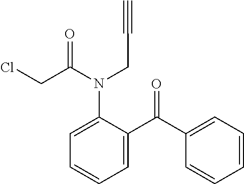 [SMILES: C#CCN(C(=O)CCl)c1ccccc1C(=O)c1ccccc1] | 0.04356 | 1.29 | 0.70 | 1.11 | 0.42 |
| 4 | IITK3004 | 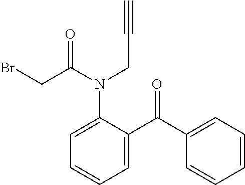 [SMILES: C#CCN(C(=O)CBr)c1ccccc1C(=O)c1ccccc1] | 0.004639 | 0.37 | | 0.13 | 0.12 |
| 5 | IITK3005 | 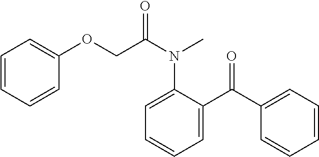 [SMILES: CN(C(=O)COc1ccccc1)c1ccccc1C(=O)c1ccccc1] | N.D. | N.D. | N.D. | N.D. | N.D. |
| 6 | IITK3006 | 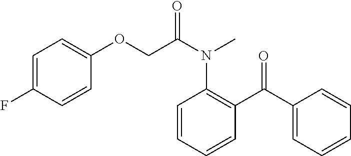 [SMILES: CN(C(=O)COc1ccc(F)cc1)c1ccccc1C(=O)c1ccccc1] | >20 | >20 | >20 | >20 | >20 |
| 7 | IITK3007 | 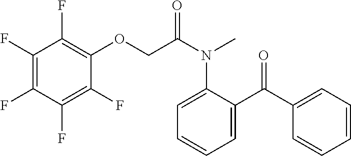 [SMILES: CN(C(=O)COc1c(F)c(F)c(F)c(F)c1F)c1ccccc1C(=O)c1ccccc1] | >20 | N.D. | N.D. | N.D. | N.D. |
| 8 | IITK3008 | 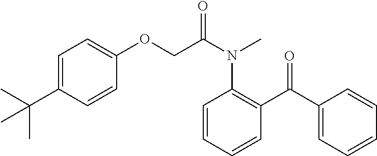 [SMILES: CN(C(=O)COc1ccc(C(C)(C)C)cc1)c1ccccc1C(=O)c1ccccc1] | >20 | N.D. | N.D. | 12.85 | N.D. |
| 9 | IITK3009 | 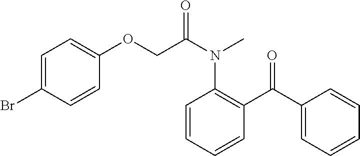 [SMILES: CN(C(=O)COc1ccc(Br)cc1)c1ccccc1C(=O)c1ccccc1] | >20 | >20 | >20 | >20 | >20 |
| 10 | IITK3010 | 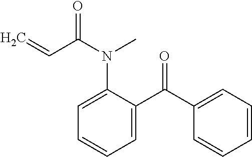 [SMILES: C=CC(=O)N(C)c1ccccc1C(=O)c1ccccc1] | N.D. | >20 | >20 | >20 | >20 |
| 11 | IITK3011 | 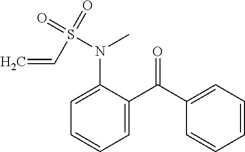 [SMILES: C=CS(=O)(=O)N(C)c1ccccc1C(=O)c1ccccc1] | N.D. | >20 | >20 | >20 | >20 |
| 12 | IITK3012 | 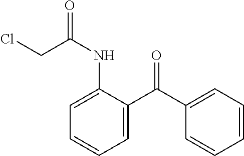 [SMILES: O=C(CCl)Nc1ccccc1C(=O)c1ccccc1] | N.D. | >20 | >20 | >20 | >20 |
| 13 | IITK3013 | 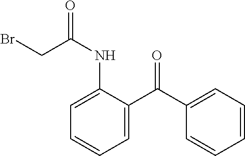 [SMILES: O=C(CBr)Nc1ccccc1C(=O)c1ccccc1] | N.D. | 1.52 | | | |
| 14 | IITK3014 | 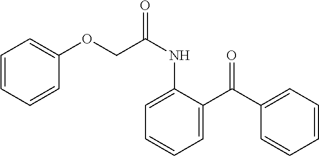 [SMILES: O=C(COc1ccccc1)Nc1ccccc1C(=O)c1ccccc1] | >20 | >20 | | | |
| 15 | IITK3018 | 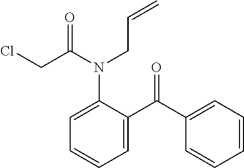 [SMILES: C=CCN(C(=O)CCl)c1ccccc1C(=O)c1ccccc1] | N.D. | 0.60 | 2.40 | N.D. | N.D. |
| 16 | IITK3019 | 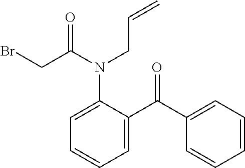 [SMILES: C=CCN(C(=O)CBr)c1ccccc1C(=O)c1ccccc1] | N.D. | 0.03 | 0.30 | N.D. | N.D. |
| 17 | IITK3020 | 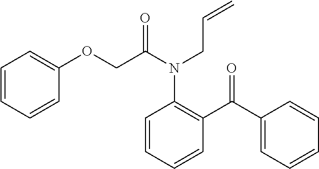 [SMILES: C=CCN(C(=O)COc1ccccc1)c1ccccc1C(=O)c1ccccc1] | N.D. | >20 | >20 | >20 | >20 |
| 18 | IITK3021 | 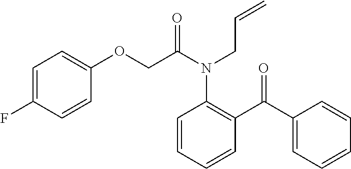 [SMILES: C=CCN(C(=O)COc1ccc(F)cc1)c1ccccc1C(=O)c1ccccc1] | N.D. | N.D. | N.D. | N.D. | N.D. |
| 19 | IITK3022 | 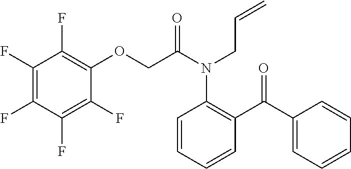 [SMILES: C=CCN(C(=O)COc1c(F)c(F)c(F)c(F)c1F)c1ccccc1C(=O)c1ccccc1] | N.D. | 1.45 | 3.37 | N.D. | N.D. |
| 20 | IITK3023 | 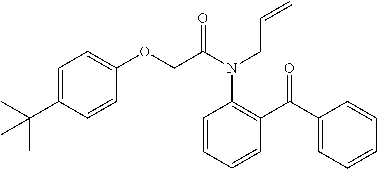 [SMILES: C=CCN(C(=O)COc1ccc(C(C)(C)C)cc1)c1ccccc1C(=O)c1ccccc1] | N.D. | >100 | 20.00 | >20 | >20 |
| 21 | IITK3024 | 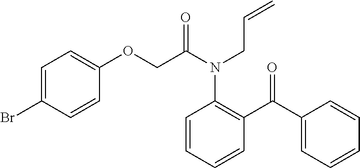 [SMILES: C=CCN(C(=O)COc1ccc(Br)cc1)c1ccccc1C(=O)c1ccccc1] | N.D. | N.D. | N.D. | N.D. | N.D. |
| 22 | IITK3025 | 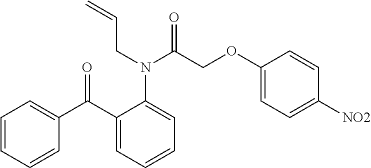 [SMILES: C=CCN(C(=O)COc1ccc([N+](=O)[O-])cc1)c1ccccc1C(=O)c1ccccc1] | >100 | N.D. | N.D. | N.D. | N.D. |
| 23 | IITK3026 | 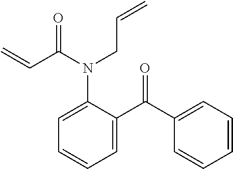 [SMILES: C=CCN(C(=O)C=C)c1ccccc1C(=O)c1ccccc1] | N.D. | N.D. | 32.82 | N.D. | N.D. |
| 24 | IITK3027 | 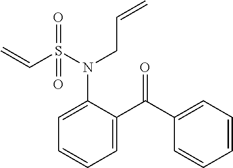 [SMILES: C=CCN(c1ccccc1C(=O)c1ccccc1)S(=O)(=O)C=C] | N.D. | 1.22 | 1.92 | N.D. | N.D. |
| 25 | IITK3028 | 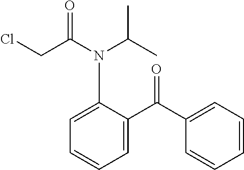 [SMILES: CC(C)N(C(=O)CCl)c1ccccc1C(=O)c1ccccc1] | 19.6 | >20 | >20 | >20 | >20 |
| 26 | IITK3029 | 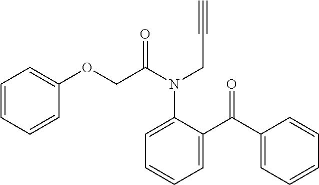 [SMILES: C#CCN(C(=O)COc1ccccc1)c1ccccc1C(=O)c1ccccc1] | >20 | >20 | >20 | >20 | >20 |
| 27 | IITK3030 | 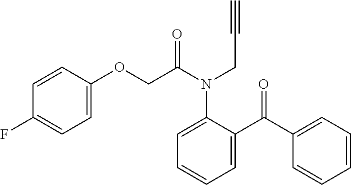 [SMILES: C#CCN(C(=O)COc1ccc(F)cc1)c1ccccc1C(=O)c1ccccc1] | >100 | >20 | >20 | >20 | >20 |
| 28 | IITK3031 | 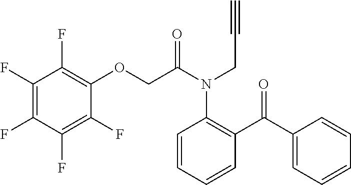 [SMILES: C#CCN(C(=O)COc1c(F)c(F)c(F)c(F)c1F)c1ccccc1C(=O)c1ccccc1] | >20 | N.D. | N.D. | N.D. | N.D. |
| 29 | IITK3032 | 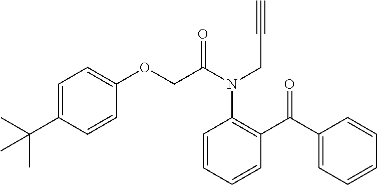 [SMILES: C#CCN(C(=O)COc1ccc(C(C)(C)C)cc1)c1ccccc1C(=O)c1ccccc1] | >20 | N.D. | N.D. | N.D. | N.D. |
| 30 | IITK3033 | 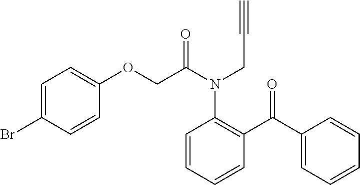 [SMILES: C#CCN(C(=O)COc1ccc(Br)cc1)c1ccccc1C(=O)c1ccccc1] | >20 | >20 | >20 | >20 | >20 |
| 31 | IITK3034 | 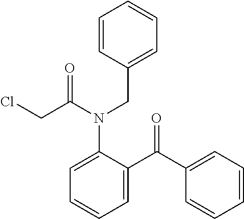 [SMILES: O=C(c1ccccc1)c1ccccc1N(Cc1ccccc1)C(=O)CCl] | N.D. | N.D. | N.D. | N.D. | 3.85 |
| 32 | IITK3035 | 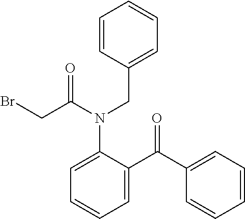 [SMILES: O=C(c1ccccc1)c1ccccc1N(Cc1ccccc1)C(=O)CBr] | N.D. | 0.19 | 0.24 | 0.53 | 0.77 |
| 33 | IITK3036 | 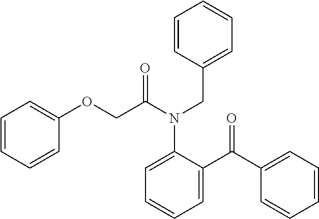 [SMILES: O=C(c1ccccc1)c1ccccc1N(Cc1ccccc1)C(=O)COc1ccccc1] | >20 | >20 | >20 | >20 | >20 |
| 34 | IITK3037 | 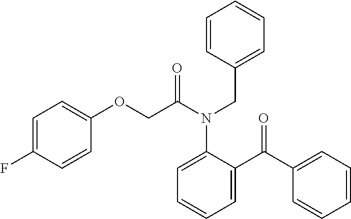 [SMILES: O=C(c1ccccc1)c1ccccc1N(Cc1ccccc1)C(=O)COc1ccc(F)cc1] | >20 | >20 | >20 | >20 | >20 |
| 35 | IITK3038 | 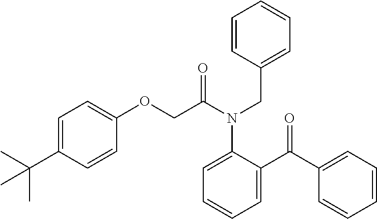 [SMILES: CC(C)(C)c1ccc(OCC(=O)N(Cc2ccccc2)c2ccccc2C(=O)c2ccccc2)cc1] | >100 | >20 | >20 | >20 | >20 |
| 36 | IITK3051 | 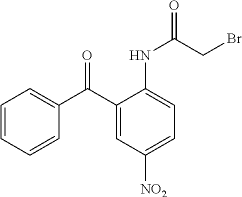 [SMILES: O=C(CBr)Nc1ccc([N+](=O)[O-])cc1C(=O)c1ccccc1] | N.D. | >20 | >20 | >20 | >20 |
| 37 | IITK3052 | 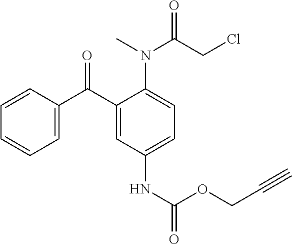 [SMILES: C#CCOC(=O)Nc1ccc(N(C)C(=O)CCl)c(C(=O)c2ccccc2)c1] | 32.76 | 2.92 | 4.18 | N.D. | N.D. |
| 38 | IITK3053 | 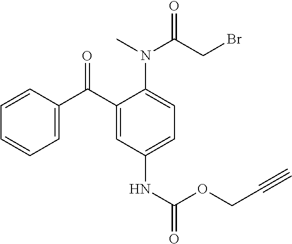 [SMILES: C#CCOC(=O)Nc1ccc(N(C)C(=O)CBr)c(C(=O)c2ccccc2)c1] | 11.74 | 0.77 | 1.54 | N.D. | N.D. |
| 39 | IITK3054 | 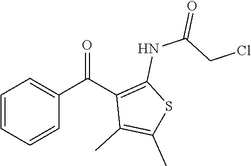 [SMILES: Cc1sc(NC(=O)CCl)c(C(=O)c2ccccc2)c1C] | N.D. | N.D. | N.D. | N.D. | N.D. |
| 40 | IITK3055 | 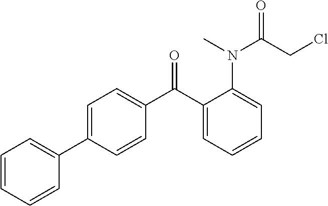 [SMILES: CN(C(=O)CCl)c1ccccc1C(=O)c1ccc(-c2ccccc2)cc1] | 0.002 | N.D. | N.D. | N.D. | N.D. |
| 41 | IITK3056 | 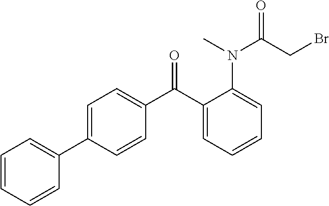 [SMILES: CN(C(=O)CBr)c1ccccc1C(=O)c1ccc(-c2ccccc2)cc1] | 0.013 | N.D. | N.D. | N.D. | N.D. |
| 42 | IITK3057 | 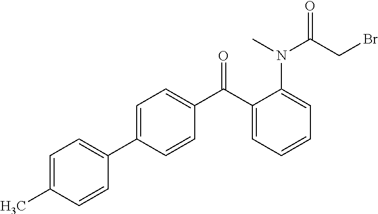 [SMILES: Cc1ccc(-c2ccc(C(=O)c3ccccc3N(C)C(=O)CBr)cc2)cc1] | 0.018 | N.D. | N.D. | N.D. | N.D. |
| 43 | IITK3058 | 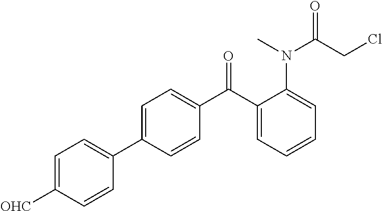 [SMILES: CN(C(=O)CCl)c1ccccc1C(=O)c1ccc(-c2ccc(C=O)cc2)cc1] | 0.117 | N.D. | N.D. | N.D. | N.D. |
| 44 | IITK3059 | 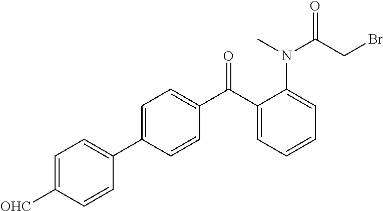 [SMILES: CN(C(=O)CBr)c1ccccc1C(=O)c1ccc(-c2ccc(C=O)cc2)cc1] | 0.079 | N.D. | N.D. | N.D. | N.D. |
| 45 | IITK3060 | 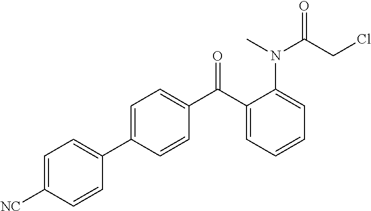 [SMILES: CN(C(=O)CCl)c1ccccc1C(=O)c1ccc(-c2ccc(C#N)cc2)cc1] | 0.026 | N.D. | N.D. | N.D. | N.D. |
| 46 | IITK3061 | 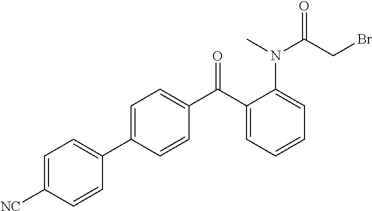 [SMILES: CN(C(=O)CBr)c1ccccc1C(=O)c1ccc(-c2ccc(C#N)cc2)cc1] | 4.824 | N.D. | N.D. | N.D. | N.D. |
| 47 | IITK3062 | 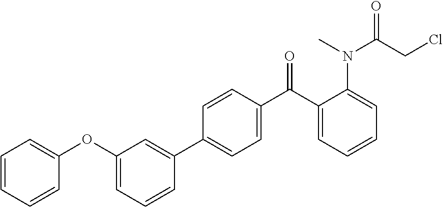 [SMILES: CN(C(=O)CCl)c1ccccc1C(=O)c1ccc(-c2cccc(Oc3ccccc3)c2)cc1] | 0.036 | N.D. | N.D. | N.D. | N.D. |
| 48 | IITK3063 | 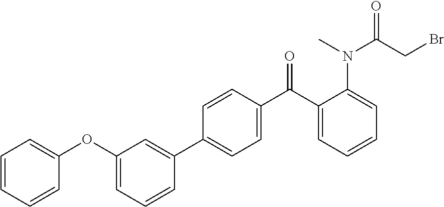 [SMILES: CN(C(=O)CBr)c1ccccc1C(=O)c1ccc(-c2cccc(Oc3ccccc3)c2)cc1] | 0.0245 | N.D. | N.D. | N.D. | N.D. |
| 49 | IITK3064 | 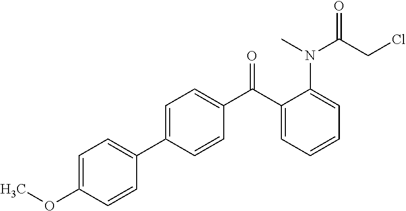 [SMILES: COc1ccc(-c2ccc(C(=O)c3ccccc3N(C)C(=O)CCl)cc2)cc1] | 0.019 | N.D. | N.D. | N.D. | N.D. |
| 50 | IITK3065 | 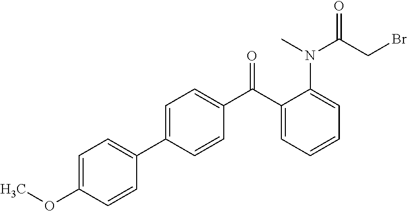 [SMILES: COc1ccc(-c2ccc(C(=O)c3ccccc3N(C)C(=O)CBr)cc2)cc1] | 0.016 | N.D. | N.D. | N.D. | N.D. |
| 51 | IITK3066 | 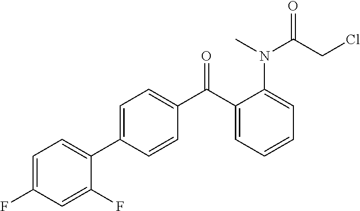 [SMILES: CN(C(=O)CCl)c1ccccc1C(=O)c1ccc(-c2ccc(F)cc2F)cc1] | 0.00883 | N.D. | N.D. | N.D. | N.D. |
| 52 | IITK3067 | 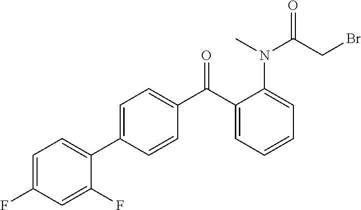 [SMILES: CN(C(=O)CBr)c1ccccc1C(=O)c1ccc(-c2ccc(F)cc2F)cc1] | 0.01211 | N.D. | N.D. | N.D. | N.D. |
| 53 | IITK3068 | 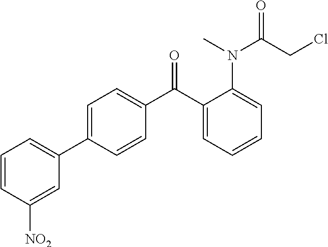 [SMILES: CN(C(=O)CCl)c1ccccc1C(=O)c1ccc(-c2cccc([N+](=O)[O-])c2)cc1] | 0.00306 | N.D. | N.D. | N.D. | N.D. |
| 54 | IITK3069 | 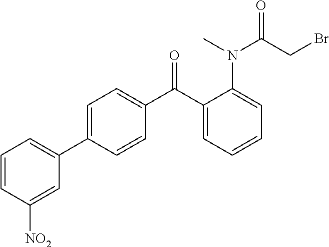 [SMILES: CN(C(=O)CBr)c1ccccc1C(=O)c1ccc(-c2cccc([N+](=O)[O-])c2)cc1] | 0.0035 | N.D. | N.D. | N.D. | N.D. |
| 55 | IITK3070 | 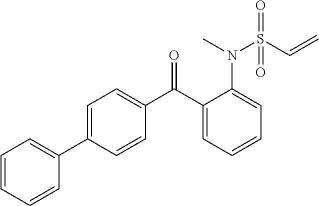 [SMILES: C=CS(=O)(=O)N(C)c1ccccc1C(=O)c1ccc(-c2ccccc2)cc1] | 8.22 | N.D. | N.D. | N.D. | N.D. |
| 56 | IITK3071 | 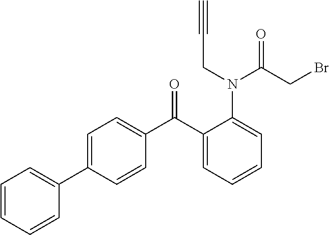 [SMILES: C#CCN(C(=O)CBr)c1ccccc1C(=O)c1ccc(-c2ccccc2)cc1] | <0.1 | N.D. | N.D. | N.D. | N.D. |
| 57 | IITK3072 | 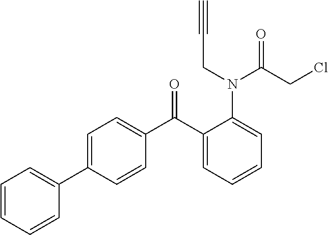 [SMILES: C#CCN(C(=O)CCl)c1ccccc1C(=O)c1ccc(-c2ccccc2)cc1] | >0.138 | N.D. | N.D. | N.D. | N.D. |
| 58 | IITK3073 | 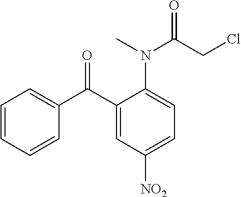 [SMILES: CN(C(=O)CCl)c1ccc([N+](=O)[O-])cc1C(=O)c1ccccc1] | >20 | N.D. | N.D. | N.D. | N.D. |

In conclusion, together our systematic SAR analysis has educated us on the sensitive and tolerable sites on our molecular scaffold for improving its potency. Of the electrophiles screened possessing diverse reactivity profiles, haloacetamides exhibited superior potency. Any modifications on the aryl-haloacetamide ring or removal/substitution of the benzoyl group were found to significantly suppress the activity of the lead scaffold. While slight modifications on the nitrogen such as allyl or propargyl were tolerated, introducing bulky functional groups or removal of substitution altogether compromised the potency of the molecule. We observed it is safe and beneficial to extend the ring system on the benzoyl group far away from the electrophile-linked aromatic system, therefore, that will be the focal point in future SAR analysis for the identification of next-generation lead molecules.

A synthesis and characterization of a library of unique organic structural scaffolds encompassing natural/unnatural amino acids, aromatic and heterocyclic linkers coupled with electrophilic units possessing diverse nucleophile reactivity profiles, and these molecules are expected to modify cellular proteins covalently, thereby modulating their functions. Also disclosed molecules exhibiting sub-micromolar to nanomolar inhibitory potency (IC₅₀) when screened against a panel of cancer cell lines including MCF7 (breast cancer), MDA-MB-231 (triple negative breast cancer), 22Rv1 (Prostate), PC3, (prostate cancer), Jurkat J6 (acute T-cell Leukaemia), RPMI 8226 (B lymphocytes), U87MG (glioblastoma), HCT-116 (colon cancer) and A375 (melanoma)) and kidney cells (HEK293). The variables of structural formula are described herein. The molecules disclosed herein are identified to promote ferroptosis in cancer cell for their mechanism of cell death action.

Therefore, the present invention provides covalent small molecules designed for targeted ferroptosis induction in cancer therapy and offers several significant advantages in the field of oncology. Firstly, these compounds demonstrate exceptional efficacy in selectively inhibiting cancer cell proliferation, with nanomolar potency observed across various cancer cell lines. Unlike traditional chemotherapies that may harm healthy cells, the specificity of ferroptosis induction minimizes collateral damage to surrounding tissues, reducing the potential for adverse side effects. Additionally, the hit-to-lead optimization strategies within the invention provide a roadmap for developing even more potent and selective lead molecules, further enhancing their therapeutic potential. Furthermore, the identification of molecular targets associated with ferroptosis induction opens new avenues for understanding the precise mechanisms behind these compounds' anticancer effects, potentially revealing novel therapeutic targets. The invention's flexibility in dosing regimens and its compatibility with combination therapies enable tailored treatment approaches, allowing for personalized cancer care. Lastly, the diagnostic applications provided by the invention facilitate the identification of patients who may benefit most from this innovative cancer therapy, paving the way for more precise and effective treatments in the fight against cancer.

While specific language has been used to describe the invention, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A 2-aminobenzophenone derivatives of Formula I, 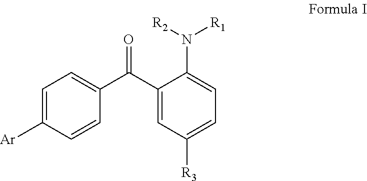
wherein,
R₁ is selected from a group consisting one of —CH₃, 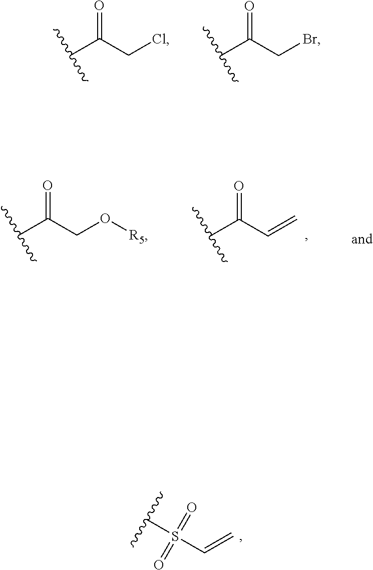
wherein R₅ is selected from a group consisting one of phenyl, 4-fluorophenyl, 4-bromophenyl, 4-tert-butylphenyl, and pentaflurophenyl;
R₂ is selected from a group consisting one of —H, methyl, allyl, benzyl, isopropyl, and propargyl;
R₃ is selected from a group consisting one of —H, —NO₂, 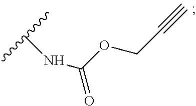 [SMILES: *NC(=O)OCC#C]
and
Ar is selected from a group consisting one of

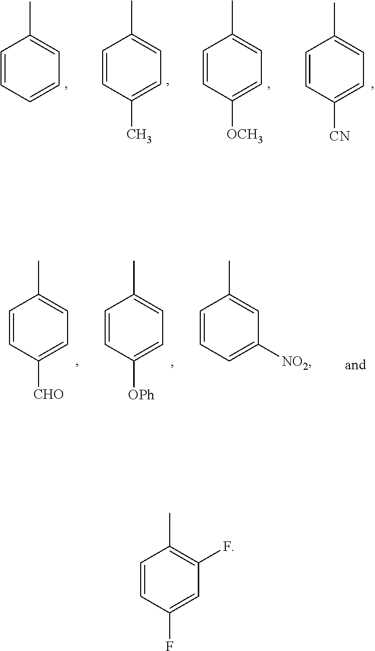

2. The 2-aminobenzophenone derivatives of Formula I as claimed in claim 1, wherein the 2-aminobenzophenone derivatives of Formula I are selected from a group consisting of N-(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), N-(2-benzoylphenyl)-2-bromo-N-(prop-2-yn-1-yl)acetamide (IITK3004), N-(2-benzoylphenyl)-N-methyl-2-phenoxyacetamide (IITK3005), N-(2-benzoylphenyl)-2-(4-fluorophenoxy)-N-methylacetamide (IITK3006), N-(2-benzoylphenyl)-N-methyl-2-(perfluorophenoxy)acetamide (IITK3007), N-(2-benzoylphenyl)-2-(4-(tert-butyl)phenoxy)-N-methylacetamide (IITK3008), N-(2-benzoylphenyl)-2-(4-bromophenoxy)-N-methylacetamide (IITK3009), N-(2-benzoylphenyl)-N-methylacrylamide (IITK3010), N-(2-benzoylphenyl)-N-methylethenesulfonamide (IITK3011), N-(2-benzoylphenyl)-2-chloroacetamide (IITK3012), N-(2-benzoylphenyl)-2-bromoacetamide (IITK3013), N-(2-benzoylphenyl)-2-phenoxyacetamide (IITK3014), N-allyl-N-(2-benzoylphenyl)-2-chloroacetamide (IITK3018), N-allyl-N-(2-benzoylphenyl)-2-bromoacetamide (IITK3019), N-allyl-N-(2-benzoylphenyl)-2-phenoxyacetamide (IITK3020), N-allyl-N-(2-benzoylphenyl)-2-(4-fluorophenoxy)acetamide (IITK3021), N-allyl-N-(2-benzoylphenyl)-2-(perfluorophenoxy)acetamide (IITK3022), N-allyl-N-(2-benzoylphenyl)-2-(4-(tert-butyl)phenoxy)acetamide (IITK3023), N-allyl-N-(2-benzoylphenyl)-2-(4-bromophenoxy)acetamide (IITK3024), N-allyl-N-(2-benzoylphenyl)-2-(4-nitrophenoxy)acetamide (IITK3025), N-allyl-N-(2-benzoylphenyl)acrylamide (IITK3026), N-allyl-N-(2-benzoylphenyl)ethenesulfonamide (IITK3027), N-(2-benzoylphenyl)-2-chloro-N-isopropylacetamide (IITK3028), N-(2-benzoylphenyl)-2-phenoxy-N-(prop-2-yn-1-yl)acetamide (IITK3029), N-(2-benzoylphenyl)-2-(4-fluorophenoxy)-N-(prop-2-yn-1-yl)acetamide (IITK3030), N-(2-benzoylphenyl)-2-(perfluorophenoxy)-N-(prop-2-yn-1-yl)acetamide (IITK3031), N-(2-benzoylphenyl)-2-(4-(tert-butyl)phenoxy)-N-(prop-2-yn-1-yl)acetamide (IITK3032), N-(2-benzoylphenyl)-2-(4-bromophenoxy)-N-(prop-2-yn-1-yl)acetamide (IITK3033), N-(2-benzoylphenyl)-N-benzyl-2-chloroacetamide (IITK3034), N-(2-benzoylphenyl)-N-benzyl-2-bromoacetamide (IITK3035), N-(2-benzoylphenyl)-N-benzyl-2-phenoxyacetamide (IITK3036), N-(2-benzoylphenyl)-N-benzyl-2-(4-fluorophenoxy)acetamide (IITK3037), N-(2-benzoylphenyl)-N-benzyl-2-(4-(tert-butyl)phenoxy)acetamide (IITK3038), N-(2-benzoyl-4-nitrophenyl)-2-bromoacetamide (IITK3051), prop-2-yn-1-yl (3-benzoyl-4-(2-chloro-N-methylacetamido)phenyl)carbamate (IITK3052), prop-2-yn-1-yl (3-benzoyl-4-(2-bromo-N-methylacetamido)phenyl)carbamate (IITK3053), N-(3-benzoyl-4,5-dimethylthiophen-2-yl)-2-chloroacetamide (IITK3054), N-(2-([1,1′-biphenyl]-4-carbonyl)phenyl)-2-chloro-N-methylacetamide (IITK3055), N-(2-([1,1′-biphenyl]-4-carbonyl)phenyl)-2-bromo-N-methylacetamide (IITK3056), 2-bromo-N-methyl-N-(2-(4′-methyl-[1,1′-biphenyl]-4-carbonyl)phenyl)acetamide (IITK3057), 2-chloro-N-(2-(4′-formyl-[1,1′-biphenyl]-4-carbonyl)phenyl)-N-methylacetamide (IITK3058), 2-bromo-N-(2-(4′-formyl-[1,1′-biphenyl]-4-carbonyl)phenyl)-N-methylacetamide (IITK3059), 2-chloro-N-(2-(4′-cyano-[1,1′-biphenyl]-4-carbonyl)phenyl)-N-methylacetamide (IITK3060), 2-bromo-N-(2-(4′-cyano-[1,1′-biphenyl]-4-carbonyl)phenyl)-N-methylacetamide (IITK3061), 2-chloro-N-methyl-N-(2-(3′-phenoxy-[1,1′-biphenyl]-4-carbonyl)phenyl)acetamide (IITK3062), 2-bromo-N-methyl-N-(2-(3′-phenoxy-[1,1′-biphenyl]-4-carbonyl)phenyl)acetamide (IITK3063), 2-chloro-N-(2-(4′-methoxy-[1,1′-biphenyl]-4-carbonyl)phenyl)-N-methylacetamide (IITK3064), 2-bromo-N-(2-(4′-methoxy-[1,1′-biphenyl]-4-carbonyl)phenyl)-N-methylacetamide (IITK3065), 2-chloro-N-(2-(2′,4′-difluoro-[1,1′-biphenyl]-4-carbonyl)phenyl)-N-methylacetamide (IITK3066), 2-bromo-N-(2-(2′,4′-difluoro-[1,1′-biphenyl]-4-carbonyl)phenyl)-N-methylacetamide (IITK3067), 2-chloro-N-methyl-N-(2-(3′-nitro-[1,1′-biphenyl]-4-carbonyl)phenyl)acetamide (IITK3068), 2-bromo-N-methyl-N-(2-(3′-nitro-[11,1-biphenyl]-4-carbonyl)phenyl)acetamide (IITK3069), N-(2-([1,1′-biphenyl]-4-carbonyl)phenyl)-N-methylethenesulfonamide (IITK3070), N-(2-([1,1′-biphenyl]-4-carbonyl)phenyl)-2-bromo-N-(prop-2-yn-1-yl)acetamide (IITK3071), N-(2-([1,1′-biphenyl]-4-carbonyl)phenyl)-2-chloro-N-(prop-2-yn-1-yl)acetamide (IITK3072), and N-(2-benzoyl-4-nitrophenyl)-2-chloro-N-methylacetamide (IITK3073).

3. The 2-aminobenzophenone derivatives of Formula I as claimed in claim 1, wherein the 2-aminobenzophenone derivatives of Formula I is selected from a group consisting one of N-(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), and N-(2-benzoylphenyl)-2-bromo-N-(prop-2-yn-1-yl)acetamide (IITK3004).

4. A pharmaceutical composition for treatment of cancer, said pharmaceutical composition comprising one or more of the 2-aminobenzophenone derivatives of Formula I, pharmaceutically acceptable salts thereof, and one of a pharmaceutically acceptable carrier, and an excipient,
wherein the pharmaceutical composition is configured for inducing ferroptosis in cancer cells.

5. The pharmaceutical composition as claimed in claim 4, wherein the 2-aminobenzophenone derivatives of Formula I is selected from a group consisting one of -(2-benzoylphenyl)-2-chloro-N-methylacetamide (IITK3001), N-(2-benzoylphenyl)-2-bromo-N-methylacetamide (IITK3002), N-(2-benzoylphenyl)-2-chloro-N-(prop-2-yn-1-yl)acetamide (IITK3003), and N-(2-benzoylphenyl)-2-bromo-N-(prop-2-yn-1-yl)acetamide (IITK3004).

6. A process for the preparation of the 2-aminobenzophenone derivatives of the Formula I, said process comprising the steps of:
reacting 2-amino-benzophenone with alkyl halide (R₆—X) in presence of 3 equivalent (equiv.) potassium carbonate (K₂CO₃) in dimethyl formamide at 90° C. for 12 hours to obtain an intermediate,
wherein R₆ is selected from a group consisting of methyl, allyl, benzyl, and propargyl; and
reacting the intermediate with 1.5 equiv. of chloroacetyl chloride in presence of 3 equiv. sodium bicarbonate (NaHCO₃) carbonate in dichloromethane at room temperature for 6 hours to obtain one of the 2-aminobenzophenone derivatives of Formula I consisting one of N-(2-benzoylphenyl)-2-chloro-N-methylacetamide (IITK3001), N-allyl-N-(2-benzoylphenyl)-2-chloroacetamide (IITK3018), N-(2-benzoylphenyl)-N-benzyl-2-chloroacetamide (IITK3034), and N-(2-benzoylphenyl)-2-chloro-N-(prop-2-yn-1-yl)acetamide (IITK3003).

7. The process for the preparation of the 2-aminobenzophenone derivatives of the Formula I as claimed in claim 6, said process further comprising the steps of:
reacting 2-amino-benzophenone with alkyl halide (R₆—X) in presence of 3 equivalent (equiv.) potassium carbonate (K₂CO₃) in dimethyl formamide at 90° C. for 12 hours to obtain the intermediate,
wherein R₆ is selected from a group consisting of methyl (2), allyl, benzyl (4), and propargyl; and
reacting the intermediate with 1.5 equiv. of bromoacetyl chloride in presence of 3 equiv. sodium bicarbonate (NaHCO₃) in dichloromethane at room temperature for 6 hours to obtain one of the 2-aminobenzophenone derivatives of Formula I consisting one of N-(2-benzoylphenyl)-2-bromo-N-methylacetamide (IITK3002), N-allyl-N-(2-benzoylphenyl)-2-bromoacetamide (IITK3019), N-(2-benzoylphenyl)-N-benzyl-2-bromoacetamide (IITK3035), and N-(2-benzoylphenyl)-2-bromo-N-(prop-2-yn-1-yl)acetamide (IITK3004).

8. The process for the preparation of the 2-aminobenzophenone derivatives of the Formula I as claimed in claim 6, said process further comprising the steps of:
reacting 2-amino-benzophenone with isopropyl bromide in presence of 3 equiv. cesium carbonate (CS₂CO₃) in dimethyl formamide at 110° C. for 12 hours to obtain an intermediate; and
reacting the intermediate with 1.5 equiv. of chloroacetyl chloride in presence of 3 equiv. sodium bicarbonate (NaHCO₃) in dichloromethane at room temperature for 6 hours to obtain one of the 2-aminobenzophenone derivatives of Formula I consisting of N-(2-benzoylphenyl)-2-chloro-N-isopropylacetamide (IITK3028).

9. The process for the preparation of the 2-aminobenzophenone derivatives of the Formula I as claimed in claim 6, said process further comprising the steps of:
reacting 2-amino-benzophenone with alkyl halide (R₆—X) in presence of 3 equiv. potassium carbonate (K₂CO₃) in dimethyl formamide at 90° C. for 12 hours to obtain the intermediate;
reacting 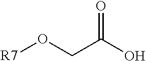
with 1.5 equiv. thionyl chloride in dichloromethane
at 0° C. to room temperature for 2 hours to obtain 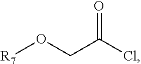
wherein R₇ is selected from a group consisting one of phenyl, 4-fluorophenyl, 4-bromophenyl, 4-tert-butylphenyl, and pentaflurophenyl; and
reacting the intermediate with the intermediate 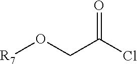
in presence of 3 equiv. sodium bicarbonate (NaHCO₃) in dichloromethane at 0° C. to room temperature for 6 hours to obtain one of the 2-aminobenzophenone derivatives of Formula I consisting one of N-(2-benzoylphenyl)-2-(4-fluorophenoxy)-N-methylacetamide (IITK3006), N-(2-benzoylphenyl)-N-methyl-2-(perfluorophenoxy)acetamide (IITK3007), N-(2-benzoylphenyl)-2-(4-(tert-butyl)phenoxy)-N-methylacetamide (IITK3008), N-(2-benzoylphenyl)-2-(4-bromophenoxy)-N-methylacetamide (IITK3009), N-(2-benzoylphenyl)-2-phenoxyacetamide (IITK3014), N-allyl-N-(2-benzoylphenyl)-2-phenoxyacetamide (IITK3020), N-allyl-N-(2-benzoylphenyl)-2-(4-fluorophenoxy)acetamide (IITK3021), N-allyl-N-(2-benzoylphenyl)-2-(perfluorophenoxy)acetamide (IITK3022), N-allyl-N-(2-benzoylphenyl)-2-(4-(tert-butyl)phenoxy)acetamide (IITK3023), N-allyl-N-(2-benzoylphenyl)-2-(4-bromophenoxy)acetamide (IITK3024), N-allyl-N-(2-benzoylphenyl)-2-(4-nitrophenoxy)acetamide (IITK3025), N-(2-benzoylphenyl)-2-phenoxy-N-(prop-2-yn-1-yl)acetamide (IITK3029), N-(2-benzoylphenyl)-2-(4-fluorophenoxy)-N-(prop-2-yn-1-yl)acetamide (IITK3030), N-(2-benzoylphenyl)-2-(perfluorophenoxy)-N-(prop-2-yn-1-yl)acetamide (IITK3031), N-(2-benzoylphenyl)-2-(4-(tert-butyl)phenoxy)-N-(prop-2-yn-1-yl)acetamide (IITK3032), and N-(2-benzoylphenyl)-2-(4-bromophenoxy)-N-(prop-2-yn-1-yl)acetamide (IITK3033).

10. The process for the preparation of the 2-aminobenzophenone derivatives of the Formula I as claimed in claim 6, said process further comprising the steps of:
reacting 2-amino-benzophenone with 1.5 equiv. methyl iodide in presence of 3 equiv. sodium bicarbonate (NaHCO₃) in dimethyl formamide at 110° C. for 12 hours to obtain the intermediate; and
reacting the intermediate with 1.5 equiv. electrophiles in presence of 3 equiv. base in dichloromethane at room temperature for 6 hours to obtain one of the 2-aminobenzophenone derivatives of Formula I consisting one of N-(2-benzoylphenyl)-N-methylacrylamide (IITK3010), and N-(2-benzoylphenyl)-N-methylethenesulfonamide (IITK3011).

11. The process for the preparation of the 2-aminobenzophenone derivatives of the Formula I as claimed in claim 6, said process further comprising the steps of:
reacting (2-amino-5-nitrophenyl)(phenyl)methanone with 1.5 equiv. methyl iodide in presence of 3 equiv. caesium carbonate (CS₂CO₃) in dimethyl formamide at 110° C. for 22 hours to obtain the intermediate 2-(methylamino)-5-nitrophenyl)(phenyl)methanone;
reacting the intermediate 2-(methylamino)-5-nitrophenyl)(phenyl) methanone with 0.1 equiv. palladium/carbon (Pd/C) in tetrahydrofuran for 3 hours at room temperature to obtain an intermediate (5-amino-2-(methylamino)phenyl)(phenyl)methanone;
reacting the intermediate (5-amino-2-(methylamino)phenyl)(phenyl) methanone with 1 equiv. Propargyloxycarbonyl-Cl (Poc-Cl) and 3 equiv. sodium bicarbonate (NaHCO₃) in tetrahydrofuran at room temperature for 3 hours to obtain an intermediate prop-2-yn-1-yl (3-benzoyl-4-(methylamino)phenyl)carbamate; and
reacting the intermediate prop-2-yn-1-yl (3-benzoyl-4-(methylamino)phenyl)carbamate with 2 equiv. triethyl amine and 1.5 equiv. electrophile in dichloromethane at room temperature for 6 hours to obtain one of the 2-aminobenzophenone derivatives of Formula I consisting one of prop-2-yn-1-yl (3-benzoyl-4-(2-chloro-N-methylacetamido) phenyl)carbamate (IITK3052), and prop-2-yn-1-yl (3-benzoyl-4-(2-bromo-N-methylacetamido) phenyl)carbamate (IITK3053),
wherein the electrophile is selected from a group consisting one of

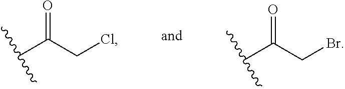 [SMILES: *C(=O)CCl ; *C(=O)CBr]

12. The process for the preparation of the 2-aminobenzophenone derivatives of the Formula I as claimed in claim 6, said process further comprising the steps of:
reacting (2-aminophenyl)(4-bromophenyl)methanone with 1.5 equiv. methyl iodide in presence of 3 equiv. potassium carbonate (K₂CO₃) in dimethyl formamide at 90° C. for 12 hours to obtain an intermediate (4-bromophenyl)(2-(methylamino)phenyl)methanone; and
reacting the intermediate (4-bromophenyl)(2-(methylamino)phenyl) methanone with 1.5 equiv. phenylboronic acid, 0.01 equiv. Palladium(II) acetate (Pd(OAc)₂) and 2 equiv. potassium carbonate (K₂CO₃) in dimethyl formamide at 80° C. for 4-6 hours to obtain compounds with formula 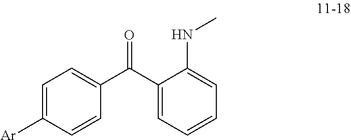
wherein,
Ar is selected from the group consisting one of

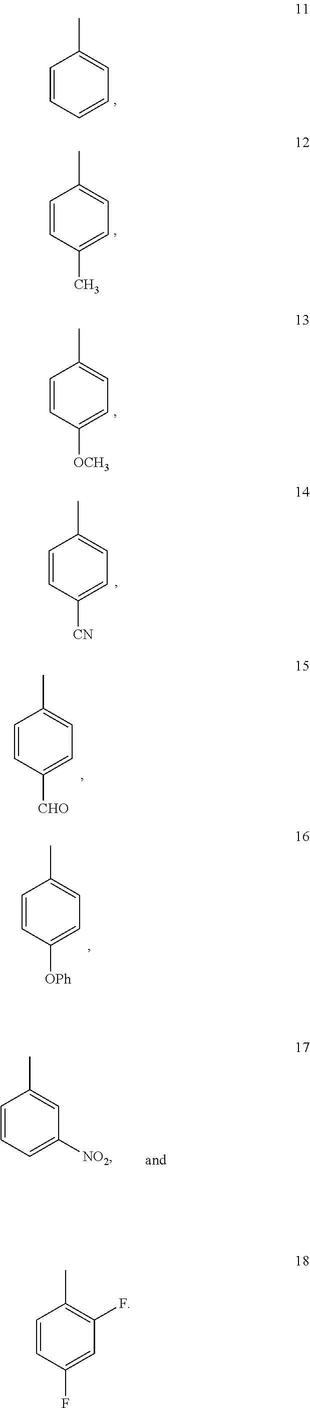

13. The process for the preparation of the 2-aminobenzophenone derivatives of the Formula I as claimed in claim 6, said process further comprising the steps of:
reacting 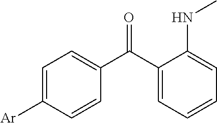
with 1.5 equiv. electrophile and 3 equiv. base in dichloromethane at room temperature for 6 hours to obtain compounds with formula 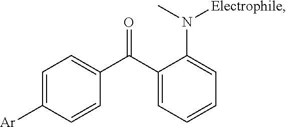
wherein the electrophile is selected from a group consisting one of

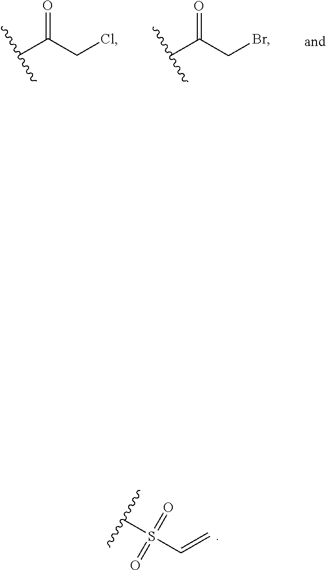

14. The process for the preparation of the 2-aminobenzophenone derivatives of Formula I as claimed in claim 13, wherein the compounds with the formula 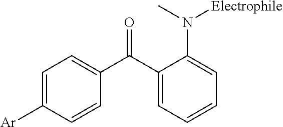
is selected from a group consisting one of N-(2-([1,1′-biphenyl]-4-carbonyl)phenyl)-2-chloro-N-methylacetamide (IITK3055), N-(2-([1,1′-biphenyl]-4-carbonyl)phenyl)-2-bromo-N-methylacetamide (IITK3056), 2-bromo-N-methyl-N-(2-(4′-methyl-[1,1′-biphenyl]-4-carbonyl)phenyl)acetamide (IITK3057), 2-chloro-N-(2-(4′-formyl-[1,1′-biphenyl]-4-carbonyl)phenyl)-N-methylacetamide (IITK3058), 2-bromo-N-(2-(4′-formyl-[1,1′-biphenyl]-4-carbonyl)phenyl)-N-methylacetamide (IITK3059), 2-chloro-N-(2-(4′-cyano-[1,1′-biphenyl]-4-carbonyl)phenyl)-N-methylacetamide (IITK3060), 2-bromo-N-(2-(4′-cyano-[1,1′-biphenyl]-4-carbonyl)phenyl)-N-methylacetamide (IITK3061), 2-chloro-N-methyl-N-(2-(3′-phenoxy-[1,1′-biphenyl]-4-carbonyl)phenyl)acetamide (IITK3062), 2-bromo-N-methyl-N-(2-(3′-phenoxy-[1,1′-biphenyl]-4-carbonyl)phenyl)acetamide (IITK3063), 2-chloro-N-(2-(4′-methoxy-[1,1′-biphenyl]-4-carbonyl)phenyl)-N-methylacetamide (IITK3064), 2-bromo-N-(2-(4′-methoxy-[1,1′-biphenyl]-4-carbonyl)phenyl)-N-methylacetamide (IITK3065), 2-chloro-N-(2-(2′,4′-difluoro-[1,1′-biphenyl]-4-carbonyl) phenyl)-N-methylacetamide (IITK3066), 2-bromo-N-(2-(2′,4′-difluoro-[1,1′-biphenyl]-4-carbonyl)phenyl)-N-methylacetamide (IITK3067), 2-chloro-N-methyl-N-(2-(3′-nitro-[1,1′-biphenyl]-4-carbonyl)phenyl) acetamide (IITK3068), 2-bromo-N-methyl-N-(2-(3′-nitro-[1,1′-biphenyl]-4-carbonyl)phenyl)acetamide (IITK3069), N-(2-([1,1′-biphenyl]-4-carbonyl)phenyl)-N-methylethenesulfonamide (IITK3070), N-(2-([1,1′-biphenyl]-4-carbonyl)phenyl)-2-bromo-N-(prop-2-yn-1-yl)acetamide (IITK3071), and N-(2-([1,1′-biphenyl]-4-carbonyl)phenyl)-2-chloro-N-(prop-2-yn-1-yl)acetamide (IITK3072).

15. The process for the preparation of the 2-aminobenzophenone derivatives of the Formula I as claimed in claim 6, said process further comprising the steps of:
reacting (2-amino-5-nitrophenyl)(phenyl)methanone with 1.5 equiv. methyl iodide in presence of 3 equiv. cesium carbonate (CS₂CO₃) in dimethyl formamide at 110° C. for 12 hours to obtain an intermediate 2-(methylamino)-5-nitrophenyl)(phenyl)methanone; and
reacting the intermediate 2-(methylamino)-5-nitrophenyl)(phenyl) methanone with 1.5 equiv. of chloroacetyl chloride in presence of 3 equiv. sodium bicarbonate (NaHCO₃) carbonate in dichloromethane at room temperature for 6 hours to obtain N-(2-benzoyl-4-nitrophenyl)-2-chloro-N-methylacetamide (IITK3073).
